(19) United States
(12) Patent Application Publication
(10) Pub. No.: US 2005/0288213 A1
(43) Pub. Date: Dec. 29, 2005

(54) Combination therapy for the treatment of obesity

(76) Inventors: Douglas J. MacNeil, Westfield, NJ (US); James H. McIntyre, Piscataway, NJ (US); Leonardus H. T. Van Der Ploeg, Scotch Plains, NJ (US); Akane Ishihara, Tsukuba, Ibaraki (JP)

Correspondence Address: MERCK AND CO., INC, P O BOX 2000, RAHWAY, NJ, 07065-0907, US

(21) Appl. No.: 10/520,566

(22) PCT Filed: Jul. 14, 2003
(86) PCT No.: PCT/US03/22077

§ 371 Date: Jan. 7, 2005

Related U.S. Application Data
(60) Provisional application No. 60/396,603, filed on Jul. 18, 2002.
(60) Provisional application No. 60/417,999, filed on Oct. 11, 2002.

(51) Int. Cl. A61K 38/17; A61K 31/4747; A61K 31/353; A61K 31/155

(52) U.S. Cl. 514/2; 514/278; 514/454; 514/635; 514/369

(57) ABSTRACT

The present invention relates to compositions comprising a NPY5 antagonist of formula I or II 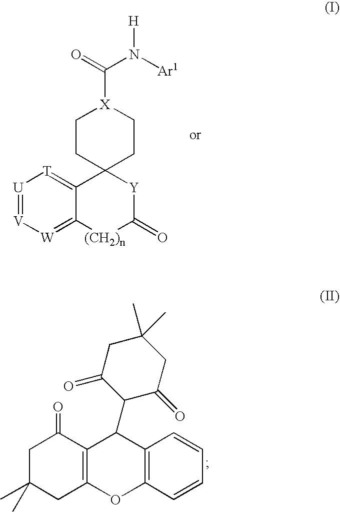 and an anti-obesity agent, useful for the treatment and prevention of diabetes, obesity and obesity-related disorders. The present invention further relates to methods of treating or preventing obesity and obesity-related disorder in a subject in need thereof by administering a composition of the present invention. The present invention further provides for pharmaceutical compositions, medicaments, and kits useful in carrying out these methods.

### BACKGROUND OF THE INVENTION

Obesity, which can be defined as a body weight more than 20% above the ideal body weight, is a major health concern in Western societies. It is estimated that about 97 million adults in the United States are overweight or obese. Obesity is the result of a positive energy balance, as a consequence of increased ratio of caloric intake to energy expenditure. The molecular factors regulating food intake and body weight balance are incompletely understood. [B. Staels et al., J. Biol. Chem. 270(27), 15958 (1995); F. Lonnquist et al., Nature Medicine 1(9), 950 (1995)]. Although the genetic and/or environmental factors leading to obesity are poorly understood, several genetic factors have been identified.

Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arterioscelerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Obesity is often treated by encouraging patients to lose weight by reducing their food intake or by increasing their exercise level and therefore increasing their energy output. A sustained weight loss of 5% to 10% of body weight has been shown to improve the co-morbidities associated with obesity, such as diabetes and hypertension, and can lead to improvement of obesity-related conditions such as osteoarthritis, sleep apnea and pulmonary and cardiac dysfunction.

Weight loss drugs that are currently used in monotherapy for the treatment of obesity have limited efficacy and significant side effects. Studies of the weight loss medications orlistat (Davidson, M. H. et al. (1999) JAMA 281:23542), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a limited weight loss of about 5%-10% of body weight for drug compared to placebo. In particular, both sibutramine and orlistat reduce body weight less than 10% over a 6 month or a 1 year period. Preclinical studies have also found that most agents, such as sibutramine, fenfluramine, Y5 antagonists, CB-1 inverse agonists and Mc4r agonists, potently inhibit food intake and decrease body weight initially. However, during chronic treatment periods of greater than 10 days the efficacy of these agents decreases yielding no more than 10% body weight loss compared to control. Obese humans can easily mass over 150 kg and would, therefore, need to lose more than 50% of their body mass to return to a normal body mass. For these patients, single agents are likely to have minimal therapeutic utility. The side effects of these drugs and anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

While single agents may be efficacious for the treatment of obesity in certain patients, due to the polygenic nature of obesity etiology, it is predicted that no single agent will be efficacious for the vast majority of obese patients. Combination therapy is more likely to achieve the desired medical benefits without the trial and error involved in prescribing each agent individually during primary care.

Commercially available combination therapies, which include phentermine as one of the components, have lead to mixed results. Phentermine was prescribed with fenfluramine (Pondimin®) or dexfenfluramine (Redux®) as a combination therapy known as fen-phen, which was withdrawn from the market in 1997 based on studies suggesting that the drugs cause damage to the mitral valve of the heart and pulmonary hypertension. Additionally, both fenfluramine and phentamine (phentermine) work through the same mechanism, namely the serotonin and norepinephrine pathway.

Due to the side effects and limited efficacy of the anti-obesity drugs currently available for mono-and combination therapy, there is a need for a combination weight loss treatment with enhanced efficacy and fewer undesirable side effects. The instant invention addresses this problem by providing a combination therapy comprised of a NPY5 antagonist and a second anti-obesity agent useful in the treatment and prevention of obesity and obesity-related disorders.

It has now been found that the combination of an NPY5 antagonist and an anti-obesity agent that decreases appetite or food intake, increases the metabolic rate or inhibits nutrient absorption, is advantageous in the treatment of obesity over treatment with either the NPY5 antagonist or the anti-obesity agent alone. The compositions of the present invention are more effective than currently available mono- and combination therapies based on the mode of action of the NPY5 antagonist and the second anti-obesity agent in these compositions. Additionally, treatment with the compositions of the present invention allow the use of the maximum efficacious dose of a NPY5 antagonist, which has no significant side effects, and a sub-clinical dose of a second anti-obesity agent, with known side effects, resulting in effective treatment with fewer side effects than current monotherapies.

It is an object of the present invention to identify compositions comprising an NPY5 antagonist and an anti-obesity agent useful for the treatment of obesity and obesity-related diseases. It is another object of the invention to identify methods of treating obesity. It is yet another object of the invention to identify methods of preventing obesity. It is a further object of the present invention to provide pharmaceutical compositions comprising a NPY5 antagonist and a second anti-obesity agent. It is yet a further object of the present invention to provide a method of manufacture of a medicament useful in the treatment of obesity.

### SUMMARY OF THE INVENTION

The present invention provides compositions comprising a NPY5 antagonist and a second anti-obesity agent useful in the treatment or prevention of obesity, and obesity-related disorders.

The present invention also provides compositions comprising a NPY5 antagonist of general Formula I or II: 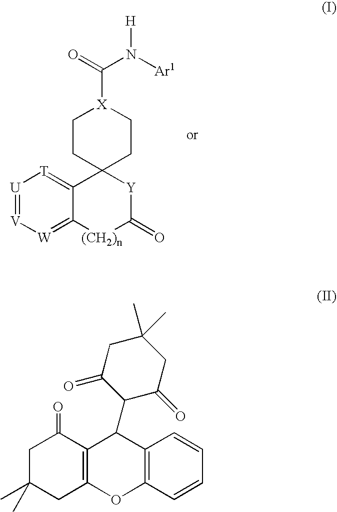
and a second anti-obesity agent useful in the treatment or prevention of obesity, and obesity-related disorders.

The present invention is further concerned with compositions comprising a NPY5 antagonist and a second anti-obesity agent selected from the group consisting of: a 5HT transporter inhibitor, a NE transporter inhibitor, a CB-1 antagonist/inverse agonist, a Ghrelin antagonist, a H3 antagonist/inverse agonist, a MCH1R antagonist, a MCH2R agonist/antagonist, a NPY1 antagonist, leptin, a leptin derivative, an opioid antagonist, an orexin antagonist, a BRS3 agonist, a CCK-A agonist, a CNTF, a CNTF derivative, a GHS agonist, a 5HT2C agonist, a Mc4r agonist, a monoamine reuptake inhibitor, an UCP-1, 2, or 3 activator, a β3 agonist, a thyroid hormone β agonist, a PDE inhibitor, a FAS inhibitor, a DGAT1 inhibitor, a DGAT2 inhibitor, an ACC2 inhibitor, a glucocorticoid antagonist, an acyl-estrogen, a lipase inhibitor, a fatty acid transporter inhibitor, a dicarboxylate transporter inhibitor, a glucose transporter inhibitor, a serotonin reuptake inhibitors, metformin, and topiramate.

The compositions of the present invention are useful in the treatment or prevention of the following obesity related disorders: overeating; bulimia; hypertension; diabetes, elevated plasma insulin concentrations; insulin resistance; dyslipidemias; hyperlipidemia; endometrial, breast, prostate and colon cancer; osteoarthritis; obstructive sleep apnea; cholelithiasis; gallstones; abnormal heart rhythms; heart arrythymias; myocardial infarction; congestive heart failure; coronary heart disease; sudden death; stroke; polycystic ovarian disease; craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g, children with acute lymphoblastic leukemia.

The present invention is also concerned with treatment of these conditions, and the use of the compositions of the present invention for manufacture of a medicament useful for treating these conditions.

The invention is also concerned with pharmaceutical compositions comprising an NPY5 antagonist and a second anti-obesity agent, as active ingredients.

The present invention is also concerned with the use of an NPY5 antagonist of Formula I or II and a second anti-obesity agent for the manufacture of a medicament for the treatment of obesity which comprises an effective amount of NPY5 antagonist of Formula I or II and an effective amount of anti-obesity agent, together or separately.

The present invention is also concerned with a product containing a NPY5 antagonist of Formula I or II and a second anti-obesity agent as a combined preparation for simultaneous, separate or sequential use in obesity.

The present invention also relates to the treatment of obesity with a combination of a NPY5 antagonist and an anti-obesity agent which may be administered separately, the invention also relates to combining separate pharmaceutical combinations into a kit form. The kit, according to this invention, comprises two separate pharmaceutical compositions: a first unit dosage form comprising a prophylactically or therapeutically effective amount of a NPY5 antagonist of Formula I or II, or a pharmaceutically acceptable salt or ester thereof, and a pharmaceutically acceptable carrier or diluent in a first unit dosage form, and a second unit dosage form comprising a prophylactically or therapeutically effective amount of a second anti-obesity agent, or a pharmaceutically acceptable salt or ester thereof, and a pharmaceutically acceptable carrier or diluent in a second unit dosage form and a container.

### BRIEF DESCRIPTION OF THE DRAWINGS

FIG. 1. Shows the effect on body weight of 30 day treatment of diet induced obese (DIO) mice with vehicle (placebo) and 100 mg/kg of compound A (administered once-daily, P.O.), compared to a group pair-fed to amount eaten by the compound A treated group.

FIG. 2. Shows the effect on daily food intake of 30 day treatment of diet induced obese (DIO) mice with vehicle (placebo) and 100 mg/kg of Compound A (administered once-daily, P.O.).

FIG. 3. Shows the effect on rectal temperature of 30 day treatment of diet induced obese (DIO) mice with vehicle (placebo) and 100 mg/kg of Compound A (administered once-daily, P.O.), compared to a group pair-fed to amount eaten by the compound A treated group.

FIG. 4. Shows the effect on body weight of 49 day treatment of non food restricted diet induced obese (DIO) mice and 10% food restricted diet induced obese (DIO) mice with vehicle (placebo) and 30 mg/kg of Compound B (administered P.O., b.i.d.).

FIG. 5. Shows the % inhibition of body weight increase of 49 day treatment of non food restricted diet induced obese (DIO) mice and 10% food restricted diet induced obese (DIO) mice with vehicle (placebo) and 30 mg/kg of Compound B (administered P.O., b.i.d.).

### DETAILED DESCRIPTION OF THE INVENTION

Neuropeptides present in the hypothalamus play a major role in mediating the control of body weight (Flier et al., Cell, 92, 437440 (1998). Neuropeptide Y (NPY), a 36 amino acid member of the pancreatic polypeptide family with widespread distribution throughout the mammalian nervous system, is another agent which has been identified as being connected with feeding behavior. Neuropeptide Y is involved in regulating eating behavior and is an extremely potent orexigenic agent [See e.g., Stanley, B. G., et al., Peptides 13: 581-587 (1992); Sahu, A. and S. P. Kalra, Trends In Endocrinology And Metabolism 4(7): 217-224 (1993)]. NPY5 antagonists have been shown to stimulate appetite in a variety of species and at different stages of development. It has been reported that the Y5 receptor is the key subtype responsible for the feeding behavior response in mammals [WO 96/16542, published Jun. 6, 1996]. Other subtypes (e.g., NPY1, NPY4) may also be involved in weight control.

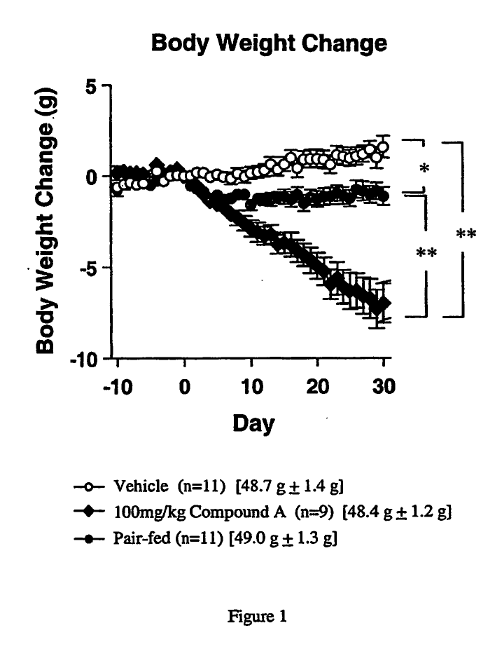

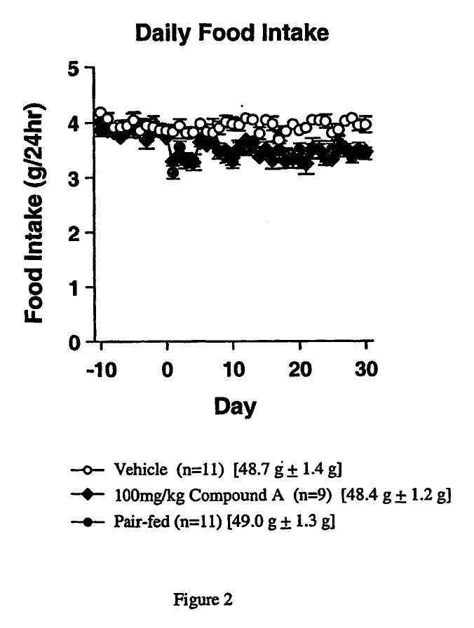

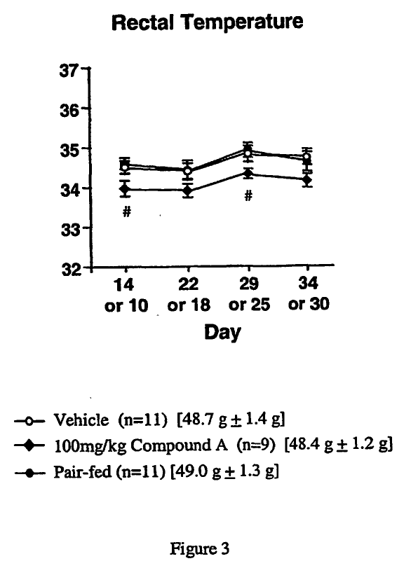

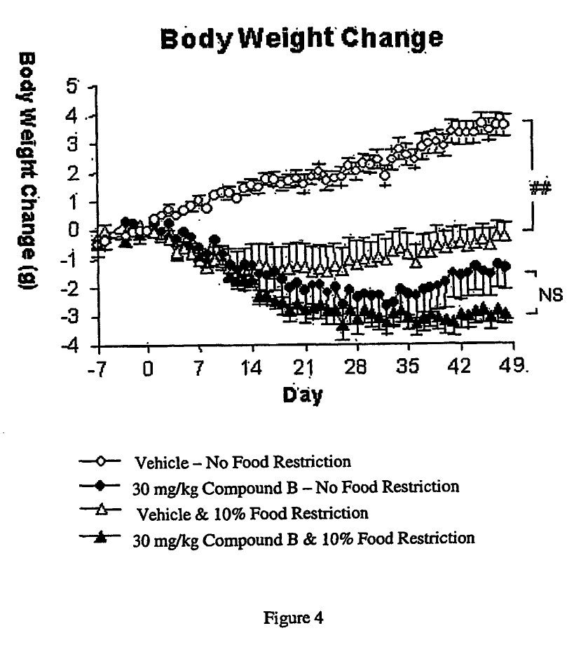

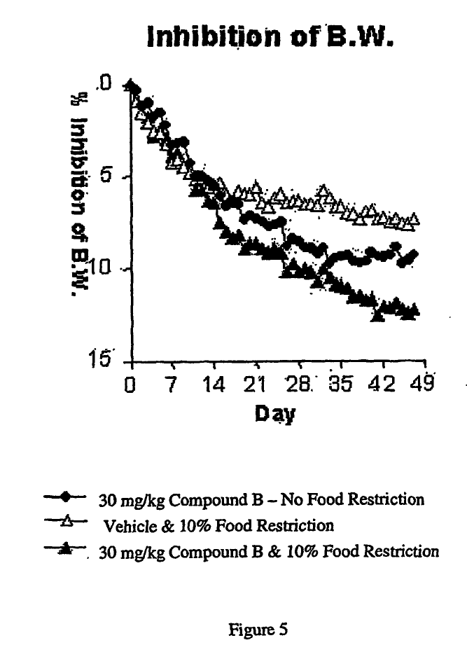

Recently, it was found that neuropeptide Y5 (NPY5) antagonists promote weight loss by a dual mechanism. First, NPY5 antagonists have been found to be mildly anorectic, leading to food intake decreases of about 4-8% in rodents. This finding is based on a study showing that diet induced obese (DIO) mice chronically treated with NPY5 antagonist (compound A) consume about 10% less food than untreated controls (See FIGS. 1 and 2). Second, NPY5 antagonists have been found to inhibit the decrease in metabolic rate observed during food restriction and dieting. This finding is supported by data showing that the pair-fed mice responded to food restriction by a compensatory decrease in body temperature, and that this drop in body temperature was prevented by treatment with the NPY5 antagonist Compound A (See FIG. 3). These data indicate that NPY5 antagonists can prevent decreases in energy expenditure and metabolic rate due to dieting or other antiobesity treatments. When DIO mice are subjected to 10% food restriction and treated with the NPY5 antagonist Compound B, they lose more weight than food restriction alone (FIGS. 4 and 5). This study further shows that a combination treatment with a NPY5 antagonist and food restriction (representing a second anti-obesity agent) results in greater weight loss than either treatment alone.

As a result of the surprising dual mode of action of NPY5 antagonists, combinations of a NPY5 antagonist and a second anti-obesity agent that affects food intake, food absorption or metabolic rate will have enhanced efficacy.

Based on the finding that NPY5 antagonists are mildly anorectic, the combination therapy with a second anti-obesity agent that decreases food intake or inhibits nutrient absorption will result in greater inhibition of food intake and caloric intake than for either agent alone. Furthermore, mouse models with enhanced metabolic rate, such as ACC2 knock-out mice (Abu-Elheiga, et al., Science 291: 2613-6 (2001)), DGAT KO knock out mice (Smith, et al., Nature Genetics, 25:87-90 (2000)), or transgenic mice over expressing UCP3 (Wolf Nutrition Rev., 59:56-7 (2001)), are resistant to diet induced obesity, however, they are often hyperphagic. Based on their anorectic properties, NPY5 antagonists can reduce the food intake, including hyperphagia associated with compounds that increase metabolic rate. As a result, the combination of a NPY5 antagonist and a second anti-obesity agent that increases metabolic rate will lead to greater efficacy in the treatment of obesity than either compound alone. Finally, based on the ability of NPY5 antagonists to inhibit the decrease in metabolic rate usually observed when food is restricted, such as during dieting, the administration of a combination of a NPY5 antagonist and a second anti-obesity agent that decreases food intake or nutrient absorption will enhance the impact of food restriction or nutrient limitation on body weight by preventing the decrease in metabolic rate usually seen after food restriction (Menozzi et al., Br. J. Nutrition. 84:515-20 (2000)). The combination of an NPY5 antagonist and a second anti-obesity agent is useful for prolonging resistance to weight gain and weight regain. Additionally, the combination of an NPY5 antagonist and a second anti-obesity agent is useful for maintaining weight loss, including weight loss due to any cause, including but not limited to diet, drug therapy and exercise.

The present invention provides compositions comprising a NPY5 antagonist and a second anti-obesity agent useful in the treatment or prevention of obesity and obesity-related disorders.

The methods and compositions of the present invention comprise a NPY5 antagonist. The NPY5 antagonists of use in the present invention may be any NPY5 antagonist known in the art. The NPY5 antagonist maybe peptidal or non-peptidyl in nature, however, the use of a non-peptidal NPY5 antagonist is preferred. For convenience, the use of an orally active NPY5 antagonist is also preferred.

The methods and compositions of the present invention comprise an anti-obesity agent. The anti-obesity agents useful in the compositions of the present invention include: a 5HT (serotonin) transporter inhibitor, a NE (norepinephrine) transporter inhibitor, a CB-1 (cannabinoid-1) antagonist/inverse agonist, a ghrelin antagonist, a H3 (histamine H3) antagonist/inverse agonist, a MCH1R (melanin concentrating hormone 1R) antagonist, a MCH2R agonist/antagonist, a NPY1 antagonist, leptin, a leptin derivative, an opioid antagonist, an orexin antagonist, a BRS3 (bombesin receptor subtype 3) agonist, a CCK-A (cholecystokinin-A) agonist, a CNTF (Ciliary neurotrophic factor), a CNTF derivative, a GHS (growth hormone secretagogue receptor) agonist, a 5HT2C (serotonin receptor 2C) agonist, a Mc4r (melanocortin 4 receptor) agonist, a monoamine reuptake inhibitor, an UCP-1 (uncoupling protein-1), 2, or 3 activator, a β3 (beta adrenergic receptor 3) agonist, a thyroid hormone β agonist, a PDE (phosphodiesterase) inhibitor, a FAS (fatty acid synthase) inhibitor, a DGAT1 (diacylglycerol acyltransferase) inhibitor, a DGAT2 inhibitor, an ACC2 (acetyl-CoA carboxylase-2) inhibitor, a glucocorticoid antagonist, an acyl-estrogen, a lipase inhibitor, a fatty acid transporter inhibitor, a dicarboxylate transporter inhibitor, a glucose transporter inhibitor, a serotonin reuptake inhibitors, metformin, and topiramate.

In one embodiment of the present invention, the NPY5 antagonists useful in the present invention are represented by the compound of structural Formula I or II: 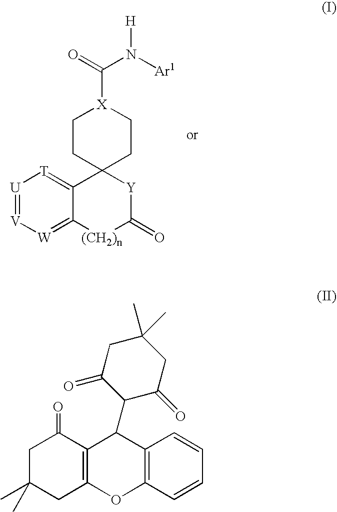
and pharmaceutically acceptable salts, esters and tautomers thereof, wherein
Ar¹ is selected from the group consisting of:
   (1) aryl, and
   (2) heteroaryl,
      wherein the aryl and heteroaryl groups are unsubstituted or optionally substituted with a substituent selected from the group consisting of:
   (a) halogen,
   (b) nitro,
   (c) lower alkyl,
   (d) halo(lower)alkyl,
   (e) hydroxy(lower)alkyl,
   (f) cyclo(lower)alkyl,
   (g) lower alkenyl,
   (h) lower alkoxy,
   (i) halo(lower)alkoxy,
   (j) lower alkylthio,
   (k) carboxyl,
   (l) lower alkanoyl,
   (m) lower alkoxycarbonyl,
   (n) lower alkylene optionally substituted with oxo, and
   (o)-Q-Ar²;
Ar² is selected from the group consisting of
   (1) aryl, and
   (2) heteroaryl,
      wherein aryl and heteroaryl are unsubstituted or optionally substituted with a substituent selected from the group consisting of:
   (a) halogen,
   (b) cyano,
   (c) lower alkyl,
   (d) halo(lower)alkyl,
   (e) hydroxy(lower)alkyl,
   (f) hydroxy,
   (g) lower alkoxy,
   (h) halo(lower)alkoxy,
   (i) lower alkylamino,
   (j) di-lower alkylamino,
   (k) lower alkanoyl, and
   (l) aryl;
n is 0 or 1;
Q is selected from the group consisting of a single bond or carbonyl;
T, U, V and W are each independently selected from the group consisting of
   (1) nitrogen, and
   (2) methine,
   wherein the methine group is unsubstituted or optionally substituted with a substituent selected from the group consisting of
   (a) halogen,
   (b) lower alkyl,
   (c) hydroxy, and
   (d) lower alkoxy; and
      wherein at least two of T, U, V, and W are methine;
X is selected from the group consisting of
   (1) nitrogen, and
   (2) methine; and
Y is selected from the group consisting of
   (1) imino, unsubstituted or optionally substituted with lower alkyl, and
   (2) oxygen.

In one class of this embodiment, the NPY5 antagonists useful in the present invention are represented by the compounds of structural Formula I: 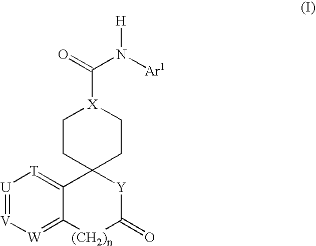
and pharmaceutically acceptable salts and esters thereof, wherein
Ar¹ is selected from the group consisting of:
   (1) aryl, and
   (2) heteroaryl,
      wherein the aryl and heteroaryl groups are unsubstituted or optionally substituted with a substituent selected from the group consisting of:
   (a) halogen,
   (b) nitro,
   (c) lower alkyl,
   (d) halo(lower)alkyl,
   (e) hydroxy(lower)alkyl,
   (f) cyclo(lower)alkyl,
   (g) lower alkenyl,
   (h) lower alkoxy,
   (i) halo(lower)alkoxy,
   (j) lower alkylthio,
   (k) carboxyl,
   (l) lower alkanoyl,
   (m) lower alkoxycarbonyl,
   (n) lower alkylene optionally substituted with oxo, and
   (o) -Q-Ar²;
Ar² is selected from the group consisting of
   (1) aryl, and
   (2) heteroaryl,
      wherein aryl and heteroaryl are unsubstituted or optionally substituted with a substituent selected from the group consisting of:
   (a) halogen,
   (b) cyano,
   (c) lower alkyl,
   (d) halo(lower)alkyl,
   (e) hydroxy(lower)alkyl,
   (f) hydroxy,
   (g) lower alkoxy,
   (h) halo(lower)alkoxy,
   (i) lower alkylamino,
   (j) di-lower alkylamino,
   (k) lower alkanoyl, and
   (l) aryl;
n is 0 or 1;
Q is selected from the group consisting of a single bond or carbonyl;
T, U, V and W are each independently selected from the group consisting of
   (1) nitrogen, and
   (2) methine,
   wherein the methine group is unsubstituted or optionally substituted with a substituent selected from the group consisting of
   (a) halogen,
   (b) lower alkyl,
   (c) hydroxy, and
   (d) lower alkoxy; and
      wherein at least two of T, U, V, and W are methine;
X is selected from the group consisting of
   (1) nitrogen, and
   (2) methine; and
Y is selected from the group consisting of
   (1) imino, unsubstituted or optionally substituted with lower alkyl, and
   (2) oxygen.

In a sub-class of this class, the NPY5 antagonist is selected from the group consisting of:
(1) N-(4-benzoylphenyl)-3-oxospiro[isoindoline-1,4′-piperidine]-1′-carboxamide,
(2) 3-oxo-N-(5-phenyl-2-pyrazinyl)spiro[isoindoline-1,4′-piperidine]-1′-carboxamide,
(3) N-(7-methyl-2-quinolyl)-3-oxospiro[isoindoline-1,4′-piperidine]-1′-carboxamide,
(4) N-(4-benzoylphenyl)-2-methyl-3-oxospiro[isoindoline-1,4′-piperidine]-1′-carboxamide,
(5) N-(4-benzoylphenyl)-3,4-dihydro-3-oxospiro[isoquinoline-1(2H),4′-piperidine]-1′-carboxamide,
(6) 3,4-dihydro-3-oxo-N-(5-phenyl-2pyrazinyl)spiro-[isoquinoline-1(2H),4′-piperidine]-1′-carboxamide,
(7) 3,4-dihydro-N-(7-methyl-2-quinolyl)-3-oxospiro-[isoquinoline-1(2H),4′-piperidine]-1′-carboxamide,
(8) N-(4-acetylphenyl)-3,4-dihydro-3-oxospiro-[isoquinoline-1(2H),4′-piperidine]-1′-carboxamide,
(9) 3,4-dihydro-3-oxo-N-[1-(2-quinolyl)-4-imidazolyl]-spiro[isoquinoline-1(2H),4′-piperidine]-1′-carboxamide,
(10) 3,4-dihydro-3-oxo-N-(5-oxo-5,6,7,8-tetrahydro-2-naphthyl)spiro[isoquinoline-1(2H),4′-piperidine]-1′-carboxamide,
(11) 3,4-dihydro-N-[5-(2-methyl-1-propenyl)-2-pyrazinyl]-3-oxospiro[isoquinoline-1(2H),4′-piperidine]-1′-carboxamide,
(12) 3,4-dihydro-3-oxo-N-(3-phenyl-5-isoxazolyl)spiro-[isoquinoline-1(2H),4′-piperidine]-1′-carboxamide,
(13) N-[1-(7-benzo[b]furanyl)-4-imidazolyl]-3,4-dihydro-3-oxospiro[isoquinoline-1(2H),4′-piperidine]-1′-carboxamide,
(14) N-[1-(3-difluoromethoxyphenyl)-4-imidazolyl]-3,4-dihydro-3-oxospiro[isoquinoline-1(2H),4′-piperidine]-1′-carboxamide,
(15) 3,4-dihydro-3-oxo-N-[4-(2-pyridylcarbonyl)phenyl]-spiro[isoquinoline-1(2H1),4′-piperidine]-1′-carboxamide,
(16) N-(3,4-dichlorophenyl)-3,4-dihydro-3-oxospiro-[isoquinoline-1(2H),4′-piperidine]-1′-carboxamide,
(17) N-[1-(3-chlorophenyl)-4-imidazolyl]-3,4-dihydro-3-oxospiro[isoquinoline-1(2H),4′-piperidine]-1′-carboxamide,
(18) 3,4-dihydro-3-oxo-N-(5-phenyl-2-thiazolyl)spiro-[isoquinoline-1(2H),4′-piperidine]-1′-carboxamide,
(19) 3,4-dihydro-3-oxo-N-[5-(2-pyridyl)-2-pyrazinyl]spiro-[isoquinoline-1(2H),4′-piperidine]-1′-carboxamide,
(20) 3,4-dihydro-N-(4-methyl-2-benzothiazolyl)-3-oxospiro-[isoquinoline-1(2H),4′-piperidine]-1′-carboxamide,
(21) N-(5-chloro-2-benzoxazolyl)-3,4-dihydro-3-oxospiro-[isoquinoline-1(2H),4′-piperidine]-1′-carboxamide,
(22) N-(4-benzoylphenyl)-3-oxospiro[isobenzofuran-1(3H),4′-piperidine]-1′-carboxamide,
(23) 3-oxo-N-(5-phenyl-2-pyrazinyl)-spiro[isobenzofuran-1(3H),4′-piperidine]-1′-carboxamide,
(24) N-(7-methyl-2-quinolyl)-3-oxospiro[isobenzofuran-1(3H),4′-piperidine]-1′-carboxamide,
(25) 3-oxo-N-(3-phenyl-5-isoxazolyl)spiro[isobenzofuran-1(3H),4′-piperidine]-1′-carboxamide,
(26) 3-oxo-N-(7-trifluoromethylpyrido[3,2-b]pyridin-2-yl)spiro[isobenzofuran-1(3H),4′-piperidine]-1′-carboxamide,
(27) 3-oxo-N-(5-phenyl-2-pyrimidinyl)spiro[isobenzofuran-1(3H),4′-piperidine]-1′-carboxamide,
(28) 3-oxo-N-[1-(3-quinolyl)-4-imidazolyl]spiro[isobenzofuran-1(3H),4′-piperidine]-1′-carboxamide,
(29) 3-oxo-N-(5-phenyl-3-pyrazolyl)spiro[isobenzofuran-1(3H),4′-piperidine]-11′-carboxamide,
(30) N-[5-(4-chlorophenyl)-3-pyrazolyl]-3-oxospiro-[isobenzofuran-1(3H),4′-piperidine]-1′-carboxamide,
(31) 3-oxo-N-[5-(3-quinolyl)-3-pyrazolyl]spiro[isobenzofuran-1(3H),4′-piperidine]-1′-carboxamide,
(32) N-[5-(3-fluorophenyl)-2-pyrimidinyl]-3-oxospiro-[isobenzofuran-1(3H),4′-piperidine]-1′-carboxamide,
(33) 3-oxo-N-[5-(3-trifluoromethylphenyl)-2-pyrimidinyl]-spiro[isobenzofuran-1(3H),4′-piperidine]-11′-carboxamide,
(34) N-[5-(3-chlorophenyl)-2-pyrimidinyl]-3-oxospiro-[isobenzofuran-1(3H),4′-piperidine]-1′-carboxamide,
(35) N-(7-difluoromethoxypyrido[3,2-b]pyridin-2-yl)-3-oxospiro[isobenzofuran-1(3H),4′-piperidine]-1′-carboxamide,
(36) 3-oxo-N-(5-phenyl-1,2,4-thiadiazol-3-yl)spiro-[isobenzofuran-1(3H),4′-piperidine]-1′-carboxamide,
(37) N-{1-[3-(2-hydroxyethyl)phenyl]4-imidazoly}-3-oxospiro-[isobenzofuran-1(3H),4′-piperidine]-1′-carboxamide,
(38) N-[4-(1-ethyl-2-imidazolyl)phenyl]-3-oxospiro-[isobenzofuran-1(3H),4′-piperidine]-1′-carboxamide,
(39) N-[1-(3-methoxyphenyl)-4-imidazolyl]-3-oxospiro-[isobenzofuran-1(3H),4′-piperidine]-1′-carboxamide,
(40) 6-fluoro-3-oxo-N-(5-phenyl-2-pyrazinyl)spiro-[isobenzofuran-1(3H),4′-piperidine]-1′-carboxamide,
(41) 6-fluoro-3-oxo-N-(5-phenyl-2-pyrimidinyl)spiro-[isobenzofuran-1(3H),4′-piperidine]-1′-carboxamide,
(42) 5-fluoro-3-oxo-N-(5-phenyl-2-pyrazinyl)spiro[isobenzofuran-1(3H),4′-piperidine]-1′-carboxamide,
(43) 5-fluoro-3-oxo-N-(5-phenyl-2-pyrimidinyl)spiro-[isobenzofuran-1(3H),4′-piperidine]-1′-carboxamide,
(44) N-(4-benzoylphenyl)-3,4-dihydro-3-oxospiro[1H-2-benzopyran-1,4′-piperidine]-1′ carboxamide,
(45) 3,4-dihydro-3-oxo-N-(5-phenyl-2-pyrazinyl)spiro[1H-2-benzopyran-1,4′-piperidine]-1′-carboxamide,
(46) N-(5-benzoyl-2-pyrazinyl)-3,4-dihydro-3-oxospiro[1H-2-benzopyran-1,4′-piperidine]-1′-carboxamide,
(47) trans-N-(4-benzoylphenyl)-3′-oxospiro[cyclohexane-1,1′(3′H)-isobenzofuran]4-carboxamide,
(48) trans-3′-oxo-N-(5-phenyl-2-pyrazinyl)spiro[cyclohexane-1,1′(3′H)-isobenzofuran]-4-carboxamide,
(49) trans-3′-oxo-N-(1-phenyl-4-imidazolyl)spiro[cyclohexane-1,1′(3′H)-isobenzofuran]-4-carboxamide,
(50) trans-3′-oxo-N-(5-phenyl-2-pyrimidinyl)spiro[cyclohexane-1,1′(3′H)-isobenzofuran]4-carboxamide,
(51) trans-N-[1-(3,5-difluorophenyl)-4-imidazolyl]-3′-oxospiro-[cyclohexane-1,1′(3′H)-isobenzofuran]-4-carboxamide,
(52) trans-3′-oxo-N-(5-phenyl-3-pyrazolyl)spiro[cyclohexane-1,1′(3′H)-isobenzofuran]4-carboxamide,
(53) trans-N-[1-(2-fluorophenyl)-4-imidazolyl]-3′-oxospiro-[cyclohexane-1,1′(3 ′H)-isobenzofuran]4-carboxamide,
(54) trans-N-(4-acetyl-3-trifluoromethylphenyl)-3′-oxospiro-[cyclohexane-1,1′(3′H)-isobenzofuran]4-carboxamide,
(55) trans-3′-oxo-N-[1-(3-quinolyl)-4-imidazolyl]-spiro[cyclohexane-1,1′(3H)-isobenzofuran]4-carboxamide,
(56) trans-N-[1-(3-cyanophenyl)-4-imidazolyl]-3′-oxospiro-[cyclohexane-1,1′(3′H)-isobenzofuran]4-carboxamide,
(57) trans-N-(4-benzoylphenyl)-3-oxospiro[4-azaisobenzofuran-1(3H),1′-cyclohexane]4′-carboxamide,
(58) trans-3-oxo-N-(5-phenyl-2-pyrazinyl)spiro[4-azaisobenzofuran-1(3H),1′-cyclohexane]4′-carboxamide,
(59) trans-3-oxo-N-(3-phenyl-5-isoxazolyl)spiro[4-azaisobenzofuran-1(3H),1′-cyclohexane]4′-carboxamide,
(60) trans-3-oxo-N-(5-phenyl-2-pyrimidinyl)spiro[4-azaisobenzofuran-1(3H),1′-cyclohexane]4′-carboxamide,
(61) trans-N-(4-benzoylphenyl)-3-oxospiro[5-azaisobenzofuran-1(3H),1′-cyclohexane]-4′-carboxamide,
(62) trans-N-(4-benzoylphenyl)-3-oxospiro[6-azaisobenzofuran-1(3H),1′-cyclohexane]-4′-carboxamide,
(63) N-[5-(4-hydroxyphenyl)-2-pyrazinyl]-3-oxospiro[isobenzofuran-1(3H),4′-piperidine]-1′-carboxamide,
(64) N-[5-(3-hydroxyphenyl)-2-pyrazinyl]-3-oxospiro[isobenzofuran-1(3H),4′-piperidine]-1′-carboxamide,
(65) 4-fluoro-3-oxo-N-(5-phenyl-2-pyrimidinyl)spiro[isobenzofuran-1(3),4′-piperidine]-1′-carboxamide,
(66) 7-fluoro-3-oxo-N-(5-phenyl-2-pyrimidinyl)spiro[isobenzofuran-1(3H),4′-piperidine]-1′-carboxamide,
(67) 6-ethyl-3-oxo-N-(5-phenyl-2-pyrazinyl)spiro[isobenzofuran-1(3H),4′-piperidine]-1′-carboxamide,
(68) 6-hydroxy-3-oxo-N-(5-phenyl-2-pyrazinyl)spiro[isobenzofuran-1(3H),4′-piperidine]-1′-carboxamide,
(69) trans-3-oxo-N-(5-phenyl-2-pyrimidinyl)spiro[5-azaisobenzofuran-1(31),1′-cyclohexane]-4′-carboxamide,
(70) trans-N-[5-(3-fluorophenyl)-2-pyrimidinyl]-3-oxospiro[5-azaisobenzofuran-1(3H1),1′-cyclohexane]-4′-carboxamide,
(71) trans-N-[5-(2-fluorophenyl)-2-pyrimidinyl]-3-oxospiro[5-azaisobenzofuran-1(3H),1′-cyclohexane]4′-carboxamide,
(72) trans-3-oxo-N-(4-phenyl-2-oxazolyl)spiro[5-azaisobenzofuran-1(3H),1′-cyclohexane]-4′-carboxamide,
(73) trans-N-[5-(2-methylphenyl)-2-pyrimidinyl]-3-oxospiro[5-azaisobenzofuran-1(3H),1′-cyclohexane]4′-carboxamide,
(74) trans-N-[5-(3-methylphenyl)-2-pyrimidinyl]-3-oxospiro[5-azaisobenzofuran-1(3H),1′-cyclohexane]-4′-carboxamide,
(75) trans-N-[5-(3-fluoromethoxyphenyl)-2-pyrimidinyl]-3-oxospiro[5-azaisobenzofuran-1(3H),1′-cyclohexane]-4′-carboxamide,
(76) trans-N-[5-(3-fluoromethylphenyl)-2-pyrimidinyl]-3-oxospiro[5-azaisobenzofuran-1(311),1′-cyclohexane]4′-carboxamide,
(77) trans-N-[5-(3-fluoro-5-methoxyphenyl)-2-pyrimidinyl]-3-oxospiro[5-azaisobenzofuran-1(3H),1′-cyclohexane]4′-carboxamide,
(78) trans-N-[5-(2-fluoro-5-methylphenyl)-2-pyrimidinyl]-3-oxospiro[5-azaisobenzofuran-1(3H),1′-cyclohexane]-4′-carboxamide,
(79) trans-N-[4-(3-fluoromethoxyphenyl)-2-oxazolyl]-3-oxospiro[5-azaisobenzofuran-1(3H),1′-cyclohexane]4′-carboxamide,
(80) trans-N-[5-(3-hydroxymethylphenyl)-2-pyrimidinyl]-3-oxospiro[5-azaisobenzofuran-1(3H),1′-cyclohexane]-4′-carboxamide,
(81) trans-N-[5-(3-hydroxyphenyl)-2-pyrimidinyl]-3-oxospiro[5-azaisobenzofuran-1(3H),1′-cyclohexane]4′-carboxamide,
(82) trans-3-oxo-N-(5-phenyl-2-pyrimidinyl)spiro[6-azaisobenzofuran-1(3H),1′-cyclohexane]4′-carboxamide,
(83) trans-N-[5-(3-fluoromethylphenyl)-2-pyrimidinyl]-3-oxospiro[6-azaisobenzofuran-1(3EQ),1′-cyclohexane]4′-carboxamide,
(84) trans-N-[5-(3-fluoromethoxyphenyl)-2-pyrimidinyl]-3-oxospiro[6-azaisobenzofuran-1(3H),1′-cyclohexane]-4′-carboxamide,
(85) trans-3-oxo-N-(6-phenyl-1,2,4-triazin-3-yl)spiro[6-azaisobenzofuran-1(311),1′-cyclohexane]-4′-carboxamide,
(86) trans-N-[5-(2-difluoromethoxyphenyl)-3-pyrazolyl]-3-oxospiro[6-azaisobenzofuran-1(3H1),1′-cyclohexane]4′-carboxamide,
(87) trans-N-[5-(3-difluoromethoxyphenyl)-3-pyrazolyl]-3-oxospiro[6-azaisobenzofuran-1(3H),1′-cyclohexane]4′-carboxamide,
(88) trans-N-[5-(3-fluorophenyl)-3-pyrazolyl]-3-oxospiro[6-azaisobenzofuran-1(311),1′-cyclohexane]-4′-carboxamide,
(89) trans-N-[5-(4-fluorophenyl)-3-pyrazolyl]-3-oxospiro[6-azaisobenzofuran-1(31),1′-cyclohexane]-4′-carboxamide,
(90) trans-N-(4-benzoylphenyl)-3-oxospiro[7-azaisobenzofuran-1(3H),1′-cyclohexane]4′-carboxamide,
(91) trans-N-[1-(3,5-difluorophenyl)-4-imidazolyl]-3-oxospiro[7-azaisobenzofuran-1(3H),1′-cyclohexane]4′-carboxamide,
(92) trans-3-oxo-N-[2-phenyl-4-pyridyl]spiro[7-azaisobenzofuran-1(3H),1′-cyclohexane]-4′-carboxamide,
(93) trans-3-oxo-N-(1-phenyl-4-pyrazolyl)spiro[7-azaisobenzofuran-1(3H),1′-cyclohexane]4′-carboxamide,
(94) trans-3-oxo-N-(1-phenyl-3-pyrrolyl)spiro[7-azaisobenzofuran-1(3H),1′-cyclohexane]-4′-carboxamide,
(95) trans-N-[1-(4-fluorophenyl)-3-pyrazolyl]-3-oxospiro[7-azaisobenzofuran-1(3H),1′-cyclohexane]-4′-carboxamide,
(96) trans-3-oxo-N-(1-phenyl-3-pyrazolyl)spiro[4-azaisobenzofuran-1(3H),1′-cyclohexane]4′-carboxamide,
(97) trans-3-oxo-N-(1-phenyl-4-pyrazolyl)spiro[4-azaisobenzofuran-1(3H),1′-cyclohexane]4′-carboxamide,
(98) trans-N-[1-(3-fluorophenyl)-4-pyrazolyl]-3-oxospiro[4-azaisobenzofuran-1(3H),1′-cyclohexane]4′-carboxamide,
(99) trans-3-oxo-N-(1-phenyl-3-pyrazolyl)spiro[6-azaisobenzofuran-1(3H),1′-cyclohexane]-4′-carboxamide,
(100) trans-N-[1-(4-fluorophenyl)-3-pyrazolyl]-3-oxospiro[6-azaisobenzofuran-1(3H),1′-cyclohexane]4′-carboxamide,
(101) trans-N-[1-(2-fluorophenyl)-3-pyrazolyl]-3-oxospiro[6-azaisobenzofuran-1(3H),1′-cyclohexane]-4′-carboxamide,
(102) trans-3-oxo-N-(5-phenyl-1,2,4-thiadiazol-3-yl)spiro[6-azaisobenzofuran-1(3H),1′-cyclohexane]-4′-carboxamide,
(103) trans-3-oxo-N-(5-phenyl-3-isoxazolyl)spiro[6-azaisobenzofuran-1(3H),1′-cyclohexane]-4′-carboxamide,
(104) trans-3-oxo-N-(6-phenyl-3-pyridyl)spiro[6-azaisobenzofuran-1(3H),1′-cyclohexane]-4′-carboxamide,
(105) trans-3-oxo-N-(2-phenyl-3-thiazolyl)spiro[6-azaisobenzofuran-1(3H),1′-cyclohexane]-4′-carboxamide,
(106) trans-3-oxo-N-(2-phenyl-1,2,3-triazol-4-yl)spiro[6-azaisobenzofuran-1(3H),1′-cyclohexane]-4′-carboxamide,
   and pharmaceutically acceptable salts and esters thereof.

In another sub-class of this class, the NPY5 antagonist is selected from the group consisting of:
(1) 3-oxo-N-(5-phenyl-2-pyrazinyl)-spiro[isobenzofuran-1(3H),4′-piperidine]-1′-carboxamide,
(2) 3-oxo-N-(7-trifluoromethylpyrido[3,2-b]pyridin-2-yl)spiro-[isobenzofuran-1(3H),4′-piperidine]-1′-carboxamide,
(3) N-[5-(3-fluorophenyl)-2-pyrimidinyl]-3-oxospiro-[isobenzofuran-1(3H),4′-piperidine]-1′-carboxamide,
(4) trans-3′-oxo-N-(5-phenyl-2-pyrimidinyl)spiro[cyclohexane-1,1′(3′H)-isobenzofuran]4-carboxamide,
(5) trans-3′-oxo-N-[1-(3-quinolyl)-4-imidazolyl]spiro[cyclohexane-1,1′(3′H)-isobenzofuran]4-carboxamide,
(6) trans-3-oxo-N-(5-phenyl-2-pyrazinyl)spiro[4-azaiso-benzofuran-1(3H),1′-cyclohexane]-4′-carboxamide,
(7) trans-N-[5-(3-fluorophenyl)-2-pyrimidinyl]-3-oxospiro[5-azaisobenzofuran-1(3H),1′-cyclohexane]-4′-carboxamide,
(8) trans-N-[5-(2-fluorophenyl)-2-pyrimidinyl]-3-oxospiro[5-azaisobenzofuran-1(3H),1′-cyclohexane]4′-carboxamide,
(9) trans-N-[1-(3,5-difluorophenyl)-4-imidazolyl]-3-oxospiro[7-azaisobenzofuran-1(3H),1′-cyclohexane]4′-carboxamide,
(10) trans-3-oxo-N-(1-phenyl-4-pyrazolyl)spiro[4-azaisobenzofuran-1(3H),1′-cyclohexane]-4′-carboxamide,
(11) trans-N-[1-(2-fluorophenyl)-3-pyrazolyl]-3-oxospiro[6-azaisobenzofuran-1(3H),1′-cyclohexane]-4′-carboxamide,
(12) trans-3-oxo-N-(1-phenyl-3-pyrazolyl)spiro[6-azaisobenzofuran-1(3H),1′-cyclohexane]-4′-carboxamide, and
(13) trans-3-oxo-N-(2-phenyl-1,2,3-triazol-4-yl)spiro[6-azaisobenzofuran-1(3H),1′-cyclohexane]-4′-carboxamide, and pharmaceutically acceptable salts and esters thereof.

In yet another sub-class of this class, the NPY5 antagonist is 3-oxo-N-(5-phenyl-2-pyrazinyl)-spiro[isobenzofuran-1(3H),4′-piperidine]-1′-carboxamide, and pharmaceutically acceptable salts and esters thereof.

In yet another sub-class of this class, the NPY5 antagonist is 3-oxo-N-(7-trifluoromethylpyrido[3,2-b]pyridin-2-yl)spiro-[isobenzofuran-1(3H),4′-piperidine]-1′-carboxamide, and pharmaceutically acceptable salts and esters thereof.

In yet another sub-class of this class, the NPY5 antagonist is N-[5-(3-fluorophenyl)-2-pyrimidinyl]-3-oxospiro-[isobenzofuran-1(3H),4′-piperidine-1′-carboxamide, and pharmaceutically acceptable salts and esters thereof.

In yet another sub-class of this class, the NPY5 antagonist is trans-3′-oxo-N-(5-phenyl-2-pyrimidinyl)spiro[cyclohexane-1,1′(3′H)-isobenzofuran]4-carboxamide, and pharmaceutically acceptable salts and esters thereof.

In yet another sub-class of this class, the NPY5 antagonist is trans-3′-oxo-N-[1-(3-quinolyl)-4-imidazolyl]spiro[cyclohexane-1,1′(3′H)-isobenzofuran]-4-carboxamide, and pharmaceutically acceptable salts and esters thereof.

In yet another sub-class of this class, the NPY5 antagonist is trans-3-oxo-N-(5-phenyl-2-pyrazinyl)spiro[4-azaiso-benzofuran-1(3H),1′-cyclohexane]-4′-carboxamide, and pharmaceutically acceptable salts and esters thereof.

In yet another sub-class of this class, the NPY5 antagonist is trans-N-[5-(3-fluorophenyl)-2-pyrimidinyl]-3-oxospiro[5-azaisobenzofuran-1(3H),1′-cyclohexane]-4′-carboxamide, and pharmaceutically acceptable salts and esters thereof.

In yet another sub-class of this class, the NPY5 antagonist is trans-N-[5-(2-fluorophenyl)-2-pyrimidinyl]-3-oxospiro[5-azaisobenzofuran-1(311),1′-cyclohexane]4′-carboxamide, and pharmaceutically acceptable salts and esters thereof.

In yet another sub-class of this class, the NPY5 antagonist is trans-N-[1-(3,5-difluorophenyl)-4-imidazolyl]-3-oxospiro[7-azaisobenzofuran-1(3H),1′-cyclohexane]-4′-carboxamide, and pharmaceutically acceptable salts and esters thereof.

In yet another sub-class of this class, the NPY5 antagonist is trans-3-oxo-N-(1-phenyl-4-pyrazolyl)spiro[4-azaisobenzofuran-1(3H),1′-cyclohexane]-4′-carboxamide, and pharmaceutically acceptable salts and esters thereof.

In yet another sub-class of this class, the NPY5 antagonist is trans-N-[1-(2-fluorophenyl)-3-pyrazolyl]-3-oxospiro[6-azaisobenzofuran-1(3H),1′-cyclohexane]-4′-carboxamide, and pharmaceutically acceptable salts and esters thereof.

In yet another sub-class of this class, the NPY5 antagonist is trans-3-oxo-N-(1-phenyl-3-pyrazolyl)spiro[6-azaisobenzofuran-1(3H),1′-cyclohexane]-4′-carboxamide, and pharmaceutically acceptable salts and esters thereof.

In yet another sub-class of this class, the NPY5 antagonist is trans-3-oxo-N-(2-phenyl-1,2,3-triazol-4-yl)spiro[6-azaisobenzofuran-1(3H)1,1′-cyclohexane]-4′-carboxamide, and pharmaceutically acceptable salts and esters thereof.

In yet another sub-class of this class, the NPY5 antagonist is 3-oxo-N-(5-phenyl-2-pyrazinyl)-spiro[isobenzofuran-1(3H),4′-piperidine]-1′-carboxamide, and pharmaceutically acceptable salts and esters thereof.

The NPY5 antagonists of formula I and their preparation are disclosed in U.S. Pat. Nos. 6,326,375; 6,335,345; and International Publication No. WO 01/14376.

In another class of the first embodiment of the present invention, the NPY5 antagonist useful in the present invention is represented by the compound of structural Formula II: 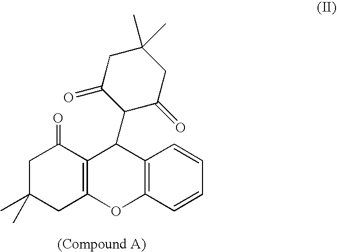
and pharmaceutically acceptable salts, esters and tautomers thereof.

The NPY5 antagonist of Formula II (Compound A) and its preparation are disclosed in J. Organic Chemistry, vol. 31, No. 5, p. 1639 (1966); and U.S. Pat. No. 6,258,837.

In another embodiment of the present invention, the anti-obesity agent is selected from a Mc4r agonist, and pharmaceutically acceptable salts and esters thereof.

In one class of this embodiment, the Mc4r agonist is selected from the group consisting of:
(1) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide,
(2) 2-[2-(1-{[(3S,4R)-1-tert-butyl-4-(2,4-difluorophenyl) pyrrolidin-3-yl]carbonyl}piperidin-4-yl)-5-fluoro-phenyl]-N-methylcarboxamide,
(3) 2-[2-(1-{[(3S,4R)-1-tert-butyl-4-(2,4-difluorophenyl) pyrrolidin-3-yl]carbonyl}piperidin-4-yl)-5-methyl-phenyl]-N-methylcarboxamide,
(4) 2-[2-(1-{[(3S,4R)-1-tert-butyl-4(2,4-difluorophenyl) pyrrolidin-3-yl]carbonyl}piperidin-4-yl)-phenyl]-N-methylcarboxamide,
(5) 2-[2-(1-{[(3S,4R)-1-tert-butyl-4-(2,4-difluorophenyl) pyrrolidin-3-yl]carbonyl}piperidin-4-yl)-4-methyl-phenyl]-N-methylcarboxamide,
(6) 2-[2-(1-{[(3S,4R)-1-tert-butyl-4-(2,4-difluorophenyl) pyrrolidin-3-yl]carbonyl}piperidin-4-yl)-4-fluoro-phenyl]-N-methylcarboxamide,
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine,
(8) 4-[2-(2-azetidin-1-yl-1(R)-methyl-2-oxoethyl)-4-chlorophenyl]-1-{[(3S,4R)-1-tert-butyl-4(2,4-difluorophenyl)pyrrolidin-3-yl]carbonyl}piperidine,
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine,
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine,
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine,
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine,
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide,
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide,
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide,
(18) N-{1-[2-(1-{[(3S,4R)-1-tert-butyl-4(2,4-difluorophenyl)pyrrolidin-3-yl]carbonyl}piperidin-4-yl)-5-chlorophenyl]ethyl}propanamide,
(19) N-{1-[2-(1-{[(3S,4R)-1-tert-butyl-4(2,4-difluoro-phenyl)pyrrolidin-3-yl]carbonyl}piperidin-4-yl)-5-chlorophenyl]ethyl}-N-methylurea,
(20) Methyl-2-[2-(1-{[(3S,4R)-1-tert-butyl-4(2,4-difluorophenyl)pyrrolidin-3-yl]carbonyl}piperidin-4-yl)-5-chlorophenyl]-2-methylpropanoate,
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide,
(22) N-{1-[2-(1-{[(3S,4R)-1-tert-butyl-4(2,4-difluoro-phenyl)pyrrolidin-3-yl]carbonyl}piperidin-4-yl)-5-fluorophenyl]ethyl}-N-methylurea,
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide,
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide,
(25) N-{(1S)-1-[2-(1-{[(3S,4R)-1-tert-butyl-4(2,4-difluorophenyl)pyrrolidin-3-yl]carbonyl}piperidin-4-yl)-5-fluorophenyl]ethyl}acetamide,
(26) N-{(1S)-1-[2-(1-{[(3S,4R)-1-tert-butyl-4-(2,4-difluorophenyl)pyrrolidin-3-yl]carbonyl}piperidin-4-yl)-5-chlorophenyl]propyl}acetamide, and
(27) N-{(1S)-1-[2-(1-{[(3S,4R)-1-tert-butyl-4(2,4-difluorophenyl)pyrrolidin-3-yl]carbonyl}piperidin-4-yl)-5-chlorophenyl]ethyl}pyrimidine-5-carboxamide,
   and pharmaceutically acceptable salts thereof.

In a sub-class of this class, the Mc4r agonist is selected from the group consisting of:
(1) N-1(1S)-1-[2-(1-{[(3S,4R)-1-tert-butyl-4-(2,4-difluorophenyl)pyrrolidin-3-yl]carbonyl lpiperidin-4-yl)-5-fluorophenyl]ethyl}acetamide,
(2) N-{(1S)-1-[2-(1-{[(3S,4R)-1-tert-butyl-4-(2,4-difluorophenyl)pyrrolidin-3-yl]carbonyl}piperidin-4-yl)-5-chlorophenyl]propyl}acetamide,
(3) N-{(1S)-1-[2-(1-{[(3S,4R)-1-tert-butyl-4-(2,4-difluorophenyl)pyrrolidin-3-yl]carbonyl}piperidin-4-yl)-5-chlorophenyl]ethyl}acetamide,
(4) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide,
(5) N-{(1S)-1-[2-(1-{[(3S,4R)-1-tert-butyl-4(2,4-difluorophenyl)pyrrolidin-3-yl]carbonyl}piperidin-4-yl)-5-chlorophenyl]ethyl}pyrimidine-5-carboxamide, and
(6) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine,
   and pharmaceutically acceptable salts thereof.

In another embodiment of the present invention, the anti-obesity agent is selected from the group consisting of:
(1) 5HT transporter inhibitor,
(2) NE transporter inhibitor,
(3) CB-1 antagonist/inverse agonist,
(4) Ghrelin antagonist,
(5) H3 antagonist/inverse agonist,
(6) MCH1R antagonist,
(7) MCH2R agonist/antagonist,
(8) NPY1 antagonist,
(9) leptin,
(10) leptin derivative,
(11) opioid antagonist,
(12) orexin antagonist,
(13) BRS3 agonist,
(14) CCK-A agonist,
(15) CNTF,
(16) CNTF derivative,
(17) GHS agonist,
(18) 5HT2C agonist;
(19) monoamine reuptake inhibitor,
(20) UCP-1, 2, and 3 activator;
(21) β3 agonist,
(22) thyroid hormone β agonist,
(23) PDE inhibitor,
(24) FAS inhibitor,
(25) DGAT1 inhibitor,
(26) DGAT2 inhibitor,
(27) ACC2 inhibitor,
(28) glucocorticoid antagonist,
(29) acyl-estrogens,
(30) lipase inhibitor;
(31) fatty acid transporter inhibitor,
(32) dicarboxylate transporter inhibitor,
(33) glucose transporter inhibitor,
(34) serotonin reuptake inhibitors,
(35) metformin, and
(36) topiramate,
   and pharmaceutically acceptable salts and esters thereof.

In one class this embodiment of the present invention, the anti-obesity agent is selected from the group consisting of:
(1) 5HT transporter inhibitor,
(2) NE transporter inhibitor,
(3) CB-1 antagonist/inverse agonist,
(4) Ghrelin antagonist,
(5) H3 antagonist/inverse agonist,
(6) MCH1R antagonist,
(7) MCH2R agonist/antagonist;
(8) opioid antagonist;
(9) BRS3 agonist;
(10) CCK-A agonist;
(11) CNTF;
(12) CNTF derivatives;
(13) GHS agonist;
(14) monoamine reuptake inhibitor,
(15) thyroid hormone 13 agonist,
(16) PDE inhibitor,
(17) FAS inhibitor,
(18) DGAT1 inhibitor,
(19) DGAT2 inhibitor,
(20) ACC2 inhibitor,
(21) glucocorticoid antagonist,
(22) acyl-estrogens,
(23) fatty acid transporter inhibitor,
(24) dicarboxylate transporter inhibitor,
(25) glucose transporter inhibitor,
(26) metformin, and
(27) topiramate,
   and pharmaceutically acceptable salts and esters thereof.

In another class of this embodiment, the anti-obesity agent is selected from the group consisting of:
(1) acyl-estrogens,
(2) CB-1 antagonist/inverse agonist,
(3) opioid antagonist,
(4) monoamine reuptake inhibitor,
(5) lipase inhibitor,
(6) leptin,
(7) CNTF;
(8) CNTF derivatives,
(9) metformin, and
(10) topiramate,
   and pharmaceutically acceptable salts and esters thereof.

In one sub-class of this class, the acyl-estrogen is selected from oleoyl-estrone, and the pharmaceutically acceptable salts thereof.

In one sub-class of this class, the monoamine reuptake inhibitor is selected from sibutramine, and the pharmaceutically acceptable salts thereof.

In another sub-class of this class, the CNTF derivative is selected from axokine, and the pharmaceutically acceptable salts thereof.

In another sub-class of this class, the lipase inhibitor is selected from orlistat, and the pharmaceutically acceptable salts thereof.

In another sub-class of this class, the CB-1 antagonist/inverse agonist is selected from rimonabant, and the pharmaceutically acceptable salts thereof.

In another sub-class of this class, the anti-obesity agent is selected from leptin, and the pharmaceutically acceptable salts thereof.

In another sub-class of this class, the opioid antagonist is selected from nalmefene, and the pharmaceutically acceptable salts thereof.

In another sub-class of this class, the anti-obesity agent is selected from topiramate, and the pharmaceutically acceptable salts thereof.

In yet another sub-class of this class, the anti-obesity agent is selected from metformin, and the pharmaceutically acceptable salts thereof. In another embodiment of the present invention, the anti-obesity agent is selected from the group consisting of:
(1) aminorex,
(2) amphechloral,
(3) amphetamine,
(4) benzphetamine,
(5) chlorphentermine,
(6) clobenzorex,
(7) cloforex,
(8) clominorex,
(9) clortermine,
(10) cyclexedrine,
(11) dexfenfluramine,
(12) dextroamphetamine,
(13) diethylpropion,
(14) diphemethoxidine,
(15) N-ethylamphetamine,
(16) fenbutrazate;
(17) fenfluramine,
(18) fenisorex,
(19) fenproporex,
(20) fludorex,
(21) fluminorex,
(22) furfurylmethylamphetamine,
(23) levamfetamine,
(24) levophacetoperane,
(25) mazindol,
(26) mefenorex;
(27) metamfepramone,
(28) methamphetamine;
(29) norpseudoephedrine,
(30) pentorex,
(31) phendimetrazine,
(32) phenmetrazine,
(33) phentermine,
(34) phenylpropanolamine, and
(35) picilorex,
   and pharmaceutically acceptable salts thereof.

In a class of this embodiment, the anti-obesity agent is selected from the group consisting of: dexfenfluramine, fenfluramine, and phentermine,

and pharmaceutically acceptable salts thereof.

In another embodiment of the present invention, the anti-obesity agent is Phytopharm compound 57 (CP 644,673).

In another embodiment of the present invention, the anti-obesity agent is selected from the group consisting of zonisamide, and pharmaceutically acceptable salts and esters thereof.

The present invention further relates to methods of treating or preventing obesity in a subject in need thereof by administering an effective amount of an NPY5 antagonist and a second anti-obesity agent. The present invention also relates to pharmaceutical compositions, and medicaments useful for carrying out these methods.

The present invention further relates to the use of an NPY5 antagonist of Formula I or II and an anti-obesity agent for the manufacture of a medicament for treatment of obesity which comprises an effective amount of NPY5 antagonist of Formula I or II and an effective amount of anti-obesity agent, together or separately.

The present invention further relates to a product containing a NPY5 antagonist of Formula I or II and an anti-obesity agent as a combined preparation for simultaneous, separate or sequential use in obesity.

NPY5 antagonists useful in the present invention, include, but are not limited to, the compounds described in: U.S. Pat. Nos. 6,140,354, 6,191,160, 6,313,298, 6,337,332, 6,329,395, and 6,340,683; European Patent Nos. EP-01010691, and EP-01044970; and PCT International Patent Publication Nos. WO 97/19682, WO 97/20820, WO 97/20821, WO 97/20822, WO 97/20823, WO 98/27063, WO 00/64880, WO 00/68197, WO 00/69849, WO 01/09120, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648. Specific NPY 5 antagonists useful in the combinations of the present invention, include, but are not limited to GW-569180A, GW-594884A, GW-587081×, GW-548118×; FR226928, FR 240662, FR252384; 1229U91, GI-264879A, CGP71683A, LY-377897, PD-160170, SR-120562A, SR-120819A and JCF-104. Additional specific NPY 5 antagonists useful in the combinations of the present invention, include, but are not limited to the compounds described in Norman et al., J. Med. Chem. 43:4288-4312 (2000).

One of ordinary skill in the art, can readily identify NPY5 antagonist compounds useful in the compositions and methods of the present invention using the methods described in WO 96/16542. NPY5 antagonists which are useful in the present invention generally have an IC₅₀ less than 1 uM in the NPY Y5 binding assay described in Kanatani et al., Biochem. Biophys. Res. Commun. 272:169-173 (2000). NPY5 antagonists which are preferred in the present invention generally have an IC₅₀ less than 100 nM in the NPY Y5 binding assay.

As used herein, the term “anti-obesity agent” includes compounds that reduce total food intake by 5 to 30%, or reduce caloric intake or selectively reduce intake of specific components of the diet such as carbohydrates or fats by 5 to 30%; compounds which, when administered to a subject, act to increase the metabolic rate of the subject, particularly those agents which increase metabolic rate by at least 5%, preferably 10%, most preferably 20% in 24 hour energy expenditure when administered to the subject; and compounds that inhibit the absorption of 10 to 50% of the nutrients.

One of ordinary skill in the art can readily identify anti-obesity agents useful in the compositions and methods of the present invention. Anti-obesity agents that decrease food intake can be evaluated in rodents according to the procedures described in: Daniels, A. J. et al., Regulatory Peptides, 106:47-54 (2002); Halaas, J. L. et. al., Science, 269: 543-546 (1995); and Strack, A. M., Obesity Research, 10:173-81 (2002). Anti-obesity agents that increase metabolic rate are routinely evaluated in rodents (Atgie, C., Comp. Biochem. Physiol. A. Mol. Integr. Physiol. 119:629-36 (1998); Himns-Hagan, J., American J. Physiology, 266:R1371-82 (1994)), and, even when inactive in rodents, are tested in additional species such as dog and monkey before ultimately being tested in humans (Connacher, A. A. et. al., Int'l J. Obesity, 16: 685-694 (1992); Connacher, A. A. et. al., Am. J. Clin. Nutr., 55: 258S-261S (1992); Connacher, A. A. et. al., Brit. Med. J., 296: 1217-1220 (1998)). The utility of anti-obesity agents that enhance metabolic rate is supported by experiments with mice, in which the RUI-beta gene has been deleted, that were shown to be resistant to diet induced obesity (D. E. Cummings et al. Nature 382: 622-626 (1996)). Anti-obesity agents that inhibit nutrient absorption can be evaluated in: Badr M. Z. and Chen, T. S., Toxicology, 34:333-40 (1985); Sorribas, V., J. Pharm. Pharmacol., 44:1030-2 (1992).

Serotonin (5HT) transport inhibitors useful in this invention include, but are not limited to, paroxetine, fluoxetine, fenfluramine, fluvoxamine, sertraline, and imipramine.

Norepinephrine (NE) transport inhibitors useful in this invention include, but are not limited to, GW 320659, despiramine, talsupram, and nomifensine.

Cannabinoid receptor 1 (CB-1) antagonist/inverse agonists useful in the present invention include: U.S. Pat. Nos. 5,532,237, 4,973,587, 5,013,837, 5,081,122, 5,112,820, 5,292,736, 5,624,941 and U.S. Pat. No. 6,028,084; and PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, and WO 02/076949; and EPO Application No. EP-658546. Specific CB-1 antagonists/inverse agonists useful in the present invention include, but are not limited to, rimonabant (Sanofi Synthelabo), and SR-147778 (Sanofi Synthelabo).

Ghrelin antagonists useful in the present invention, include: PCT Application Nos. WO 01/87335, and WO 02/08250.

Histamine 3 (H3) antagonist/inverse agonists useful in the present invention include: PCT Application No. WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:333543 (2000)). Specific H3 antagonists/inverse agonists useful in the present invention include, but are not limited to, thioperamide, 3-(1H-imidazol-4-yl)propyl N-(4-pentenyl)carbamate, clobenpropit, iodophenpropit, imoproxifan, and GT2394 (Gliatech).

Melanin-concentrating hormone 1 receptor (MCH1R) antagonists and melanin-concentrating hormone 2 receptor (MCH2R) agonist/antagonists useful in the present invention include PCT Patent Application Nos. WO 01/82925, WO 01/87834, WO 02/06245, WO 02/04433, and WO 02/51809; and Japanese Patent Application No. JP 13226269. Specific MCH1R antagonists useful in the present invention include, but are not limited to, T-226296 (Takeda).

Neuropeptide Y1 (NPY1) antagonists useful in the present invention, include: U.S. Pat. No. 6,001,836; and PCT Application Nos. WO 96/14307, WO 01/23387, WO 99/51600, WO 01/85690, WO 01/85098, WO 01/85173, and WO 01/89528. Specific examples of NPY1 antagonists useful in the present invention include, but are not limited to, BIB3P3226, J-115814, BIBO 3304, LY-357897, CP-671906, and GI-264879A.

Leptin includes, but is not limited to, recombinant human leptin (PEG-OB, Hoffman La Roche) and recombinant methionyl human leptin (Amgen). Leptin derivatives (e.g., truncated forms of leptin) useful in the present invention include: U.S. Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520.

Opioid antagonists useful in the present invention include: PCT Application No. WO 00/21509. Specific opioid antagonists useful in the present invention include, but are not limited to, nalmefene (Revex®), 3-methoxynaltrexone naloxone, and naltrexone.

Orexin antagonists useful in the present invention include: PCT Patent Application Nos. WO 01/96302, WO 01/68609, WO 02/51232, and WO 02/51838. Specific orexin antagonists useful in the present invention include, but are not limited to, SB-334867-A.

An acyl-estrogen useful in the present invention include oleoyl-estrone (del Mar-Grasa, M. et al., Obesity Research, 9:202-9 (2001)).

Cholecystokinin-A (CCK-A) agonists useful in the present invention include U.S. Pat. No. 5,739,106. Specific CCK-A agonists include, but are not limited to, AR-R 15849, GI 181771, JMV-180, A-71378, A-71623 and SR146131.

Specific ciliary neurotrophic factors (CNTF) useful in the present invention include, but are not limited to, GI-181771 (Glaxo-SmithKline); SR146131 (Sanofi Synthelabo); butabindide; PD170,292, PD 149164 Pfizer). CNTF derivatives useful in the present invention include, but are not limited to, axokine (Regeneron); and PCT Application Nos. WO 94/09134, WO 98/22128, and WO 99/43813.

Growth hormone secretagogue (GHS) agonists useful in the present invention include: U.S. Pat. No. 6,358,951, and U.S. Patent Application Nos. 2002/049196 and 2002/022637; and PCT Application Nos. WO 01/56592, and WO 02/32888. Specific GHS agonists include, but are not limited to, NN703, hexarelin, MK-0677, SM-130686, CP-424,391, L-692,429 and L-163,255.

5HT2C agonists useful in the present invention include: U.S. Pat. No. 3,914,250; and PCT Application Nos. WO 02/36596, WO 02/48124, WO 02/10169, WO 01/66548, WO 02/44152; WO 02/51844, WO 02/40456, and WO 02/40457. Specific 5 HT2C agonists useful in this invention include, but are not limited to, BVT933, DPCA37215, WAY161503, and R-1065.

Mc4r agonists useful in the present invention include: PCT Application Nos. WO 01/991752, WO 01/74844, WO 02/12166, WO 02/11715, WO 02/12178, WO 99/64002, WO 00/74679, WO 01/70708, WO 01/70337, WO 01/91752, WO 02/059095, WO 02/059107, WO 02/059108, WO 02/059117, WO 02/068387, WO 02/068388, WO 03/007949, and WO 03/009847. Specific Mc4r agonists useful in the present invention include CHIR86036 (Chiron); ME-10142, and ME-10145 (Melacure).

Monoamine reuptake inhibitors useful in the present invention include: PCT Application Nos. WO 01/27068, and WO 01/62341. Specific monoamine reuptake inhibitors useful in the present invention include, but are not limited to, sibutramine (Meridia®/Reductil®) disclosed in U.S. Pat. Nos. 4,746,680, 4,806,570, and 5,436,272, and U.S. Patent Publication No. 2002/0006964. The present invention encompasses sibutratmine as a racemic mixture, as optically pure isomers (+) and (−), or a pharmaceutically acceptable salt, solvent, hydrate, clathrate or prodrug thereof; particularly sibutramine hydrochloride monohydrate.

Serotonin reuptake inhibitors useful in the present invention include: U.S. Patent Application No. 6,365,633; and PCT Patent Application Nos. WO 01/27060, and WO 01/162341.

Uncoupling Protein (UCP-1, UCP-2, and UCP-3) activators useful in the present invention include: PCT Patent Application No. WO 99/00123. Specific uncoupling protein (UCP-1, UCP-2, and UCP-3) activators useful in the present invention include, but are not limited to, phytanic acid, 4-1(E)-2-(5,6,7,8-tetrahydro-5,5,8,8-tetramethyl-2-napthalenyl)-1-propenyl]benzoic acid (TTNPB), and retinoic acid.

β3 adrenergic receptor (β3) agonists useful in the present invention include: U.S. Pat. No. 5,705,515, and U.S. Pat. No. 5,451,677; and PCT Patent Application Nos. WO 01/4782, and WO 02/32897. Specific β3 agonists useful in the present invention include, but are not limited to, AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A.

Thyroid hormone β agonists useful in the present invention include: PCT Application No. WO 02/15845; and Japanese Patent Application No. JP 2000256190. Specific thyroid hormone β agonists useful in the present invention include, but are not limited to, KB-2611 (KaroBioBMS).

Specific fatty acid synthase (FAS) inhibitors useful in the present invention, include, but are not limited to, Cerulenin and C75.

Specific phosphodieterase (PDE) inhibitors useful in the present invention, include, but are not limited to, theophylline, pentoxifylline, zaprinast, sildenafil, amrinone, milrinone, cilostamide, rolipram, and cilomilast.

Lipase inhibitors useful in the present invention include: PCT Application No. WO 01/77094. Specific lipase inhibitors useful in the present invention include, but are not limited to, orlistat (Xenical®), Triton WR1339, RHC80267, lipstatin, tetrahydrolipstatin, teasaponin, and diethylumbelliferyl phosphate.

Topiramate (Topimax®), indicated as an anti-convulsant and an anti-convulsant, has been shown to increase weight loss.

Metformin (Glucophage®) is indicated for patients with non-insulin dependent diabetes mellitus, particularly those with refractory obesity. Physician's Desk Reference® page 1080-1086, (56^{th} ed. 2002).

Specific bombesin (BRS-3) agonists useful in the present invention, include, but are not limited to, [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75).

Zonisamide, a marketed antiepileptic drug with serotonergic and dopaminergic activity in addition to the ability to block sodium and calcium channels, has been shown to result in weight loss in epileptic adults and in obese adults.

The above compounds are only illustrative of the NPY5 antagonists and anti-obesity agents that can be used in the compositions of the present invention. As this listing of compounds is not meant to be comprehensive, the methods of the present invention may employ any NPY5 antagonist of formula I and any anti-obesity agent, and are not limited to any particular structural class of compounds.

The NPY5 antagonist Compound A is the compound of Formula II: 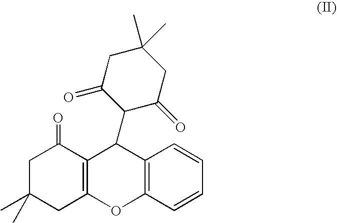
and pharmaceutically acceptable salts, esters and tautomers thereof.

The NPY5 antagonist Compound B is 3-oxo-N-(5-phenyl-2-pyrazinyl)-spiro[isobenzofuran-1(3H),4′-piperidine]-1′-carboxamide or a pharmaceutically acceptable salt or ester thereof.

The present invention also relates to the treatment of obesity with a combination of a NPY5 antagonist and an anti-obesity agent which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of a NPY5 antagonist, or a pharmaceutically acceptable salt or ester thereof, and a pharmaceutically acceptable carrier or diluent in a first unit dosage form, and a second unit dosage form comprising a prophylactically or therapeutically effective amount of another anti-obesity agent, or a pharmaceutically acceptable salt or ester thereof, and a pharmaceutically acceptable carrier or diluent in a second unit dosage form. The kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

“Halogen atom” refers to fluorine atom, chlorine atom, bromine atom and iodine atom.

“Lower alkyl” refers to a straight- or branched-chain alkyl group of C1 to C6, for example, methyl, ethyl, propyl, isopropyl, butyl, isobutyl, sec-butyl, tert-butyl, pentyl, isopentyl, hexyl, isohexyl, and the like.

“Halo(lower)alkyl” refers to the aforesaid lower alkyl substituted with 1 or more than 2, preferably 1 to 3 aforesaid halogen atoms identically or differently at the substitutable, arbitrary positions, for example, fluoromethyl, difluoromethyl, trifluoromethyl, 2-fluoroethyl, 1,2-difluoroethyl, chloromethyl, 2-chloroethyl, 1,2-dichloroethyl, bromomethyl, iodomethyl, and the like.

“Hydroxy(lower)alkyl” refers to the aforesaid lower alkyl substituted with 1 or more than 2, preferably 1 or 2 hydroxy groups at the substitutable, arbitrary positions, for example, hydroxymethyl, 2-hydroxyethyl, 1-hydroxy-1-methylethyl, 1,2-dihydroxyethyl, 3-hydroxypropyl, and the like.

“Cyclo(lower)alkyl” refers to a cycloalkyl group of C3 to C6, for example, cyclopropyl, cyclobutyl, cyclopentyl, cyclohexyl, and the like.

“Lower alkenyl” refers to a straight- or branched-chain alkenyl group of C2 to C6, for example, vinyl, 1-propenyl, 2-propenyl, isopropenyl, 3-butenyl, 2-butenyl, 1-butenyl, 1-methyl-2-propenyl, 1-methyl-1-propenyl, 1-ethyl-1-ethenyl, 2-methyl-2-propenyl, 2-methyl-1-propenyl, 3-methyl-2-butenyl, 4-pentenyl, and the like.

“Lower alkoxy” refers to a straight- or branched-chain alkoxy group of C1 to C6, for example, methoxy, ethoxy, propoxy, isopropoxy, butoxy, sec-butoxy, isobutoxy, tert-butoxy, pentyloxy, isopentyloxy, hexyloxy, isohexyloxy, and the like.

“Halo(lower)alkoxy” refers to the aforesaid lower alkoxy substituted with 1 or more than 2, preferably 1 to 3 aforesaid halogen atoms identically or differently at the substitutable, arbitrary positions, for example, fluoromethoxy, difluoromethoxy, trifluoromethoxy, 2-fluoroethoxy, 1,2-difluoroethoxy, chloromethoxy, 2-chloroethoxy, 1,2-dichloroethoxy, bromomethoxy, iodomethoxy, and the like.

“Lower alkylthio” refers to a straight- or branched-chain alkylthio group of C1 to C6, for example, methylthio, ethylthio, propylthio, isopropylthio, butylthio, sec-butylthio, isobutylthio, tert-butylthio, pentylthio, isopentylthio, hexylthio, isohexylthio, and the like.

“Lower alkanoyl” refers to an alkanoyl group containing the aforesaid lower alkyl, that is, an alkanoyl group of C2 to C7, for example acetyl, propionyl, butyryl, isobutyryl, valeryl, isovaleryl, pivaloyl, and the like.

“Lower alkoxycarbonyl” refers to an alkoxycarbonyl group containing the aforesaid lower alkoxy, that is, an alkoxycarbonyl group of C2 to C7, for example, methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, and the like.

“Lower alkylene optionally substituted with oxo” refers to a straight- or branched-chain alkylene group of C2 to C6 which may be substituted with 1 or more than 2, preferably 1 oxo group at a substitutable, arbitrary position, for example, ethylene, trimethylene, tetramethylene, pentamethylene, hexamethylene, 1-oxoethylene, 1-oxotrimethylene, 2-oxotrimethylene, 1-oxotetramethylene, 2-oxotetramethylene, and the like.

“Aryl” includes phenyl, naphthyl, and the like.

“Heteroaryl” refers to 5- or 6-membered monocylic heteroaromatic group which contains 1 or more than 2, preferably 1 to 3 hetero atoms identically or differently selected from the group of oxygen atom, nitrogen atom and sulfur atom; or condensed heteroaromatic group, where the aforesaid monocylic heteroaromatic group is condensed with the aforesaid aryl group, or with the identified or different aforesaid monocylic heteroaromatic group each other, for example, pyrrolyl, furyl, thienyl, imidazolyl, pyrazolyl, thiazolyl, isothiazolyl, oxazolyl, isoxazolyl, 1,2,3-triazolyl, 1,2,4-triazolyl, tetrazolyl, oxadiazolyl, 1,2,3-thiadiazolyl, 1,2,4 thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzoxazolyl, benzisoxazolyl, benzothiazolyl, benzisothiazolyl, indazolyl, purinyl, quinolyl, isoquinolyl, phthalazyl, naphthylidinyl, quinoxalinyl, quinazolinyl, cinnolinyl, pteridinyl, pyrido[3,2-b]pyridyl, and the like.

“Lower alkylamino” refers to an amino group mono-substituted with the aforesaid lower alkyl, for example, methylamino, ethylamino, propylamino, isopropylamino, butylamino, sec-butylamino, tert-butylamino, and the like.

“Di-lower alkylamino” refers to an amino group di-substituted with identical or different aforesaid lower alkyl, for example, dimethylamino, diethylamino, ethylmethylamino, dipropylamino, methylpropylamino, diisopropylamino, and the like.

In order to disclose the aforesaid compounds of the general formula (I) more detailed, the various symbols used in the formula (I) are explained in more detail by the use of preferred embodiments.

Ar¹ represents aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar².

“Aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar²” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or more than 2, preferably 1 or 2 selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group of formula: -Q-Ar².

Halogen atom as the aforesaid substituent includes fluorine atom, chlorine atom, and the like preferably.

Lower alkyl as the aforesaid substituent includes methyl, ethyl, propyl, isopropyl, and the like preferably.

Halo(lower)alkyl as the aforesaid substituent includes difluoromethyl, trifluoromethyl, and the like preferably.

Hydroxy(lower)alkyl as the aforesaid substituent includes hydroxymethyl, 2-hydroxyethyl, 1-hydroxy-1-methylethyl, and the like preferably.

Cyclo(lower)alkyl as the aforesaid substituent includes cyclopropyl, cyclobutyl, and the like preferably.

Lower alkenyl as the aforesaid substituent includes vinyl, 1-propenyl, 2-methyl-1-propenyl, and the like preferably.

Lower alkoxy as the aforesaid substituent includes methoxy, ethoxy, and the like preferably.

Halo(lower)alkoxy as the aforesaid substituents includes fluoromethoxy, difluoromethoxy, trifluoromethoxy, and the like preferably.

Lower alkylthio as the aforesaid substituent includes methylthio, ethylthio, and the like preferably.

Lower alkanoyl as the aforesaid substituent includes acetyl, propionyl, and the like preferably.

Lower alkoxycarbonyl as the aforesaid substituent includes methoxycarbonyl, ethoxycarbonyl, and the like preferably.

Lower alkylene optionally substituted with oxo as the aforesaid substituent includes 1-oxotetramethylene, and the like preferably.

In a group of formula: -Q-Ar² as the aforesaid substituent, Ar² represents aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
Q represents a single bond or carbonyl.

“Aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or not less than 2, preferably 1 or 2 selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl.

Halogen atom as the aforesaid substituent includes, preferably, fluorine atom, chlorine atom, and the like.

Lower alkyl as the aforesaid substituent includes, preferably, methyl, ethyl, propyl, isopropyl, and the like.

Halo(lower)alkyl as the aforesaid substituent includes, preferably, difluoromethyl, trifluoromethyl, and the like.

Hydroxy(lower)alkyl as the aforesaid substituent includes, preferably, hydroxymethyl, 2-hydroxyethyl, 1-hydroxy-1-methylethyl, and the like.

Lower alkoxy as the aforesaid substituent includes, preferably, methoxy, ethoxy, and the like.

Halo(lower)alkoxy as the aforesaid substituent includes, preferably, fluoromethoxy, difluoromethoxy, trifluoromethoxy, and the like.

Lower alkylamino as the aforesaid substituent includes, preferably, methylamino, ethylamino, and the like.

Di-lower alkylamino as the aforesaid substituent includes, preferably, dimethylamino, diethylamino, and the like.

Lower alkanoyl as the aforesaid substituent includes, preferably, acetyl, propionyl, and the like.

Aryl as the aforesaid substituent includes, preferably, phenyl, and the like.

The substituent(s) of Ar² include, preferably, halogen, cyano, lower alkyl, halo(lower)alkyl, hydroxy(lower)alkyl, hydroxy, halo(lower)alkoxy, and the like.

Aryl in Ar² includes, preferably, phenyl, and the like and heteroaryl includes imidazolyl, pyridyl, benzofuranyl, quinolyl, and the like. Consequently, a group of formula: -Q-Ar² includes, for example, phenyl, 2-fluorophenyl, 3-fluorophenyl, 4-fluorophenyl, 2,3-difluorophenyl, 2,4-difluorophenyl, 3,5-difluorophenyl, 2-chlorophenyl, 3-chlorophenyl, 4-chlorophenyl, 2-cyanophenyl, 3-cyanophenyl, 4-cyanophenyl, 2-methylphenyl, 3-methylphenyl, 4-methylphenyl, 2-fluoro-5-methylphenyl, 3-fluoromethylphenyl, 2-trifluoromethylphenyl, 3-trifluoromethylphenyl, 4-trifluoromethylphenyl, 2-methoxyphenyl, 3-methoxyphenyl, 4-methoxyphenyl, 3-fluoro-5-methoxyphenyl, 3-fluoromethoxyphenyl, 3-difluoromethoxyphenyl, 3-(2-hydroxyethyl)phenyl, 3-hydroxymethylphenyl, 3-(1-hydroxy-1-methylethyl)phenyl, 3-hydroxyphenyl, 4-hydroxyphenyl, 2-imidazolyl, 1-ethyl-2-imidazolyl, 1,2,4-thiadiazol-5-yl, 1,3,4-thiadiaol-2-yl, 2-pyridyl, 3-pyridyl, 4-pyridyl, 2-thyl-4-pyridyl, 4-pyrimidinyl, 5-pyrimidinyl, 4-benzo[b]furanyl, 5-benzo[b]furanyl, 7-benzo[b]furanyl, 2-quinolyl, 3-quinolyl, 4-quinolyl, 5-quinolyl, 6-quinolyl, 8-quinolyl, benzoyl, 2-pyridylcarbonyl, and the like, and preferably, phenyl, 2-fluorophenyl, 3-fluorophenyl, 3,5-difluorophenyl, 3-chlorophenyl, 4-chlorophenyl, 3-cyanophenyl, 3-trifluoromethylphenyl, 3-difluoromethoxyphenyl, 3-(2-hydroxyethyl)phenyl, 3-hydroxyphenyl, 4-hydroxyphenyl, 1-ethyl-2-imidazolyl, 2pyridyl, 7-benzo[b]furanyl, 2-quinolyl, 3-quinolyl, benzoyl, 2-pyridylcarbonyl, and the like.

The substituent of Ar¹ includes, preferably, halogen, lower alkyl, halo(lower)alkyl, lower alkenyl, lower alkanoyl, lower alkylene optionally substituted with oxo, and a group of formula: -Q-Ar², and the like.

Aryl in Ar¹ includes, preferably, phenyl, and the like and heteroaryl of Ar¹ includes pyrrolyl, imidazolyl, pyrazolyl, thiazolyl, oxazolyl, isoxazolyl, 1,2,3-triazolyl, 1,2,4-thiadiazolyl, pyridyl, pyrazinyl, pyrimidinyl, 1,2,4-triazinyl, benzoxazolyl, benzothiazolyl, quinolyl, pyrido[3,2-b]pyridyl, and the like. Consequently, Ar¹ includes, for example, 3-fluorophenyl, 4-fluorophenyl, 3,4-difluorophenyl, 3-chlorophenyl, 4-chlorophenyl, 3,4-dichlorophenyl, 4-acetylphenyl, 5-oxo-5,6,7,8-tetrahydro-2-naphthyl, 4-acetyl-3-trifluoromethylphenyl, 4-(1-ethyl-2-imidazolyl)phenyl, 3-(2-pyridyl)phenyl, 3-(4-pyridyl)phenyl, 4-(2-pyridyl)phenyl, 4-(3-pyridyl)phenyl, 4-(2-ethyl-4-pyridyl)phenyl, 4-(4-pyrimidinyl)phenyl, 4-benzoylphenyl, 4-(2-pyridylcarbonyl)phenyl, 1-phenyl-3-pyrrolyl, 1-phenyl-4-imidazolyl, 1-(2-fluorophenyl)-4-imidazolyl, 1-(3-fluorophenyl)-4-imidazolyl, 1-(4-fluorophenyl)-4-imidazolyl, 1-(2,3-difluorophenyl)-4-imidazolyl, 1-(2,4-difluorophenyl)-4-imidazolyl, 1-(3,5-difluorophenyl)-4-imidazolyl, 1-(3-chlorophenyl)-4-imidazolyl, 1-(2-cyanophenyl)-4-imidazolyl, 1-(3-cyanophenyl)-4-imidazolyl, 1-(4-cyanophenyl)-4-imidazolyl, 1-(3-trifluoromethylphenyl)-4-imidazolyl, 1-[3-(2-hydroxyethyl)phenyl]4-imidazolyl, 1-[3-(1-hydroxy-1-methylethyl)phenyl]-imidazolyl, 1-(3-methoxyphenyl)-4-imidazolyl, 1-(2-difluoromethoxyphenyl)-4-imidazolyl, 1-(3-difluoromethoxyphenyl)-4-imidazolyl, 1-(4-difluoromethoxy-phenyl)-4-imidazolyl, 1-(2-pyridyl)-4-imidazolyl, 1-(4-benzo[b]furanyl)-4-imidazolyl, 1-(5-benzo[b]furanyl)-4-imidazolyl, 1-(7-benzo[b]furanyl)-4-imidazolyl, 1-(2-quinolyl)-4-imidazolyl, 1-(3-quinolyl)-4-imidazolyl, 1-(4-quinolyl)-4-imidazolyl, 1-(5-quinolyl)-4-imidazolyl, 1-(6-quinolyl)-4-imidazolyl, 1-(8-quinolyl)-4-imidazolyl, 1-phenyl-3-pyrazolyl, 5-phenyl-3-pyrazolyl, 1-phenyl-4-pyrazolyl, 1-(2-fluorophenyl)-3-pyrazolyl, 5-(2-fluorophenyl)-3-pyrazolyl, 5-(3-fluorophenyl)-3-pyrazolyl, 1-(3-fluorophenyl)-4-pyrazolyl, 1-(4-fluorophenyl)-3-pyrazolyl, 5-(4-fluorophenyl)-3-pyrazolyl, 5-(2-chlorophenyl)-3-pyrazolyl, 5-(3-chlorophenyl)-3-pyrazolyl, 5-(4-chlorophenyl)-3-pyrazolyl, 5-(2-difluoromethoxyphenyl)-3-pyrazolyl, 5-(3-difluoromethoxyphenyl)-3-pyrazolyl, 2-methyl-5-phenyl-3-pyrazolyl, 5-(2-pyridyl)-3-pyrazolyl, 5-(2-quinolyl)-3-pyrazolyl, 5(3-quinolyl)-3-pyrazolyl, 4-phenyl-2-thiazolyl, 5-phenyl-2-thiazolyl, 5-(3-chlorophenyl)-2-thiazolyl, 5-(4-chlorophenyl)-2-thiazolyl, 5-(4-methoxyphenyl)-2-thiazolyl, 5-(2-pyridyl)-2-thiazolyl, 2-phenyl-4-thiazolyl, 4-phenyl-2-oxazolyl, 5-phenyl-2-oxazolyl, 4-(2-fluoromethoxyphenyl)-2-oxazolyl, 4-(3-fluoromethoxyphenyl)-2-oxazolyl, 5-phenyl-3-isoxazolyl, 3-phenyl-5-isoxazolyl, 3-(2-chlorophenyl)-5-isoxazolyl, 3-(3-chlorophenyl)-5-isoxazolyl, 3-(4-chlorophenyl)-5-isoxazolyl, 3-(2-pyridyl)-5-isoxazolyl, 2-phenyl-1,2,3-triazol-4-yl, 5-phenyl-1,2,4-thiadiazol-3-yl, 5-phenyl-1,3,4-thiadiazol-2-yl, 5-(3-chlorophenyl)-1,3,4-thiadiazol-2-yl, 5-(2-pyridyl)-1,3,4-thiadiazol-2-yl, 5-(2-ethyl-4-pyridyl)-1,3,4-thiadiazol-2-yl, 5-phenyl-2-pyridyl, 6-phenyl-3-pyridyl, 2-phenyl-4-pyridyl, 5-(2-pyridyl)-2-pyridyl, 5-benzoyl-2-pyridyl, 6-benzoyl-3-pyridyl, 5-chloro-2-pyrazinyl, 5-(2-methyl-1-propenyl)-2-pyrazinyl, 5-acetyl-2-pyrazinyl, 5-propionyl-2-pyrazinyl, 5-phenyl-2-pyrazinyl, 5-(3-hydroxyphenyl)-2-pyrazinyl, 5-(4-hydroxyphenyl)-2-pyrazinyl, 5-(1,2,4-thiadiazol-5-yl)-2-pyrazinyl, 5-(1,3,4-thiadiazol-2-yl)-2-pyrazinyl, 5-(2-pyridyl)-2-pyrazinyl, 5-(3-pyridyl)-2-pyrazinyl, 5-(5-pyrimidinyl)-2-pyrazinyl, 5-(3-quinolyl)-2-pyrazinyl, 5-benzoyl-2-pyrazinyl, 5-(2-pyridylcarbonyl)-2-pyrazinyl, 5-acetyl-2-pyrimidinyl, 5-acetyl-3-methyl-2-pyrimidinyl, 4-phenyl-2-pyrimidinyl, 5-phenyl-2-pyrimidinyl, 6-phenyl-4-pyrimidinyl, 2-phenyl-5-pyrimidinyl, 5-(2-fluorophenyl)-2-pyrimidinyl, 5-(3-fluorophenyl)-2-pyrimidinyl, 5-(4-fluorophenyl)-2-pyrimidinyl, 5-(2-chlorophenyl)-2-pyrimidinyl, 5-(3-chlorophenyl)-2-pyrimidinyl, 5-(4-chlorophenyl)-2-pyrimidinyl, 5-(2-methylphenyl)-2-pyrimidinyl, 5-(3-methylphenyl)-2-pyrimidinyl, 5-(2-fluoromethylphenyl)-2-pyrimidinyl, 5-(3-fluoromethylphenyl)-2-pyrimidinyl, 5-(2-trifluoromethylphenyl)-2-pyrimidinyl, 5-(3-trifluoromethylphenyl)-2-pyrimidinyl, 5-(4-trifluoromethylphenyl)-2-pyrimidinyl, 5-(2-hydroxymethylphenyl)-2-pyrimidinyl, 5-(3-hydroxymethylphenyl)-2-pyrimidinyl, 5-(2-hydroxyphenyl)-2-pyrimidinyl, 5-(3-hydroxyphenyl)-2-pyrimidinyl, 5-(2-methoxyphenyl)-2-pyrimidinyl, 5-(3-methoxyphenyl)-2-pyrimidinyl, 5-(4-methoxyphenyl)-2-pyrimidinyl, 5-(2-fluoromethoxyphenyl)-2-pyrimidinyl, 5-(3-fluoromethoxyphenyl)-2-pyrimidinyl, 5-(2-fluoro-5-methylphenyl)-2-pyrimidinyl, 5-(3-fluoro-5-methoxyphenyl)-2-pyrimidinyl, 6-phenyl-3-pyridazinyl, 6-phenyl-1,2,4-triazin-3-yl, 5-chloro-2-benzoxazolyl, 5-fluoro-2-benzothiazolyl, 4-methyl-2-benzothiazolyl, 2-methyl-5-benzothiazolyl, 4-methoxy-2-benzothiazolyl, 3-quinolyl, 6-quinolyl, 7-methyl-2-quinolyl, 2-methyl-6-quinolyl, 6-chloro-2-quinoxalinyl, pyrido[3,2-b]pyridin-2-yl, 7-chloropyrido[3,2-b]pyridin-2-yl, 7-methylpyrido[3,2b]pyridin-2-yl, 7-trifluoromethylpyrido[3,2-b]pyridin-2-yl, 7-difluoromethoxypyrido[3,2-b]pyridin-2-yl, 7-acetylpyrido[3,2-b]pyridin-2-yl, and the like, preferably 3,4-dichlorophenyl, 4-acetylphenyl, 5-oxo-5,6,7,8-tetrahydro-2-naphthyl, 4-acetyl-3-trifluoromethylphenyl, 4-(1-ethyl-2-imidazolyl)phenyl, 4-benzoylphenyl, 4-(2-pyridylcarbonyl)phenyl, 1-phenyl-3-pyrrolyl, 1-phenyl-4-imidazolyl, 1-(2-fluorophenyl)-4-imidazolyl, 1-(3,5-difluorophenyl)-4-imidazolyl, 1-(3-chlorophenyl)-4-imidazolyl, 1-(3-cyanophenyl)-4-imidazolyl, 1-[3-(2-hydroxyethyl)phenyl]4-imidazolyl, 1-(3-difluoromethoxyphenyl)-4-imidazolyl, 1-(7-benzo[b]furanyl)-4-imidazolyl, 1-(2-quinolyl)-4-imidazolyl, 1-(3-quinolyl)-4-imidazolyl, 1-phenyl-3-pyrazolyl, 5-phenyl-3-pyrazolyl, 1-phenyl-4-pyrazolyl, 1-(3-fluorophenyl)-4-pyrazolyl, 1-(4-fluorophenyl)-3-pyrazolyl, 5-(4-chlorophenyl)-3-pyrazolyl, 5-(3-quinolyl)-3-pyrazolyl, 5-phenyl-2-thiazolyl, 3-phenyl-5-isoxazolyl, 5-(2-methyl-1-propenyl)-2-pyrazinyl, 5-phenyl-2-pyrazinyl, 5-(3-hydroxyphenyl)-2-pyrazinyl, 5-(4-hydroxyphenyl)-2-pyrazinyl, 5-(2-pyridyl)-2-pyrazinyl, 5-benzoyl-2-pyrazinyl, 5-phenyl-2-pyrimidinyl, 5-(2-fluorophenyl)-2-pyrimidinyl, 5-(3-fluorophenyl)-2-pyrimidinyl, 5-(3-chlorophenyl)-2-pyrimidinyl, 5-(3-trifluoromethylphenyl)-2-pyrimidinyl, 5-chloro-2-benzoxazolyl, 4-methyl-2-benzothiazolyl, 7-methyl-2-quinolyl, 7-trifluoromethylpyrido[3,2-b]pyridin-2-yl, and the like, especially 1-phenyl-3-pyrazolyl, 5-phenyl-3-pyrazolyl, 5-phenyl-2-pyrazinyl, 5-(3-20; hydroxyphenyl)-2-pyrazinyl, 5-(4-hydroxyphenyl)-2-pyrazinyl, 5-phenyl-2-pyrimidinyl, 5-(2-fluorophenyl)-2-pyrimidinyl, 5-(3-fluorophenyl)-2-pyrimidinyl, 7-trifluoro-methylpyrido[3,2-b]pyridin-2-yl, and the like.

n represents 0 or 1, 0 is preferable.

T, U, V and W represent independently nitrogen atom or methine which may have a substituent selected from the group consisting of halogen, lower alkyl, hydroxy and lower alkoxy, where at least two of them represent the said methine group.

“Methine which may have a substituent selected from the group consisting of halogen, lower alkyl, hydroxy and lower alkoxy” refers to unsubstituted methine or methine having a substituent which can be selected from the group consisting of halogen, lower alkyl, hydroxy and lower alkoxy.

Halogen atom as the aforesaid substituent includes preferably fluorine atom, chlorine atom, and the like.

Lower alkyl as the aforesaid substituent includes preferably methyl, ethyl, and the like.

Lower alkoxy as the aforesaid substituent includes preferably methoxy, ethoxy, and the like.

The aforesaid substituent include preferably halogen, and the like.

The preferred mode of T, U, V and W includes, for example, T, U, V and W are independently methine optionally having the aforesaid substituent, preferably halogen; or one of T, U, V and W is nitrogen atom.

X represents methine or nitrogen.

Y represents imino which may be substituted with lower alkyl, or oxygen.

“Imino which may be substituted with lower alkyl” refers to unsubstituted imino or imino substituted with lower alkyl.

The aforesaid lower alkyl includes, preferably, methyl, ethyl, and the like.

Y is preferably unsubstituted imino or oxygen, especially oxygen.

The term “pharmaceutically acceptable salts” refers to the pharmaceutically acceptable and common salts, for example, a base addition salt to carboxyl group when the compound has a carboxyl group, or an acid addition salt to amino or basic heterocyclyl when the compound has an amino or basic heterocyclyl group, including quaternary ammonium salts, prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, trifluoro acetate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to the NPY5 antagonists, NPY5 antagonists of Formula I, NPY5 antagonists of Formula II, and anti-obesity agents are meant to also include the pharmaceutically acceptable salts and esters thereof.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The “pharmaceutically acceptable esters” in the present invention refer to non-toxic esters, for example, the pharmaceutically acceptable, common esters on carboxyl group when the compound has a carboxyl group, for example, esters with lower alkyls (for example methyl, ethyl, propyl, isopropyl, butyl, sec-butyl, tert-butyl, pentyl, isopentyl, neopentyl, cyclopropyl, cyclobutyl, cyclopentyl), aralkyls (for example benzyl, phenethyl), lower alkenyls (for example allyl. 2-butenyl), lower alkoxy (lower) alkyls (for example methoxymethyl. 2-methoxyethyl. 2-ethoxyethyl), lower alkanoyloxy (lower) alkyls (for example acetoxymethyl, pivaloyloxy-methyl. 1-pivaloyloxyethyl), lower alkoxycarbonyl (lower) alkyls (for example methoxycarbonylmethyl, isopropoxycarbonylmethyl), carboxy-(lower)alkyls (for example carboxymethyl), lower alkoxycarbonyloxy-(lower)alkyls (for example 1-(ethoxycarbonyloxy)ethyl. 1-(cyclohexyl-oxycarbonyloxy)ethyl), carbamoyloxy(lower)alkyls (for example carbamoyloxymethyl), phthalidyl group, (5-substituted-2-oxo-1,3-dioxol-4-yl)methyl (for example (5-methyl-2-oxo-1,3-dioxol-4-yl)methyl), and the like.

The compounds in the compositions of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, or tautomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of obesity and obesity-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower-back pain, gallbladder disease, gout, kidney cancer, and increased anesthetic risk. The compositions of the present invention are also useful for reducing the risk of secondary outcomes of obesity, such as reducing the risk of left ventricular hypertrophy. The compositions of the present invention are also useful to treat Alzheimer's disease.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The terms “administration of” and or “administering a” compound should be understood to mean providing a compound of the invention or a prodrug of a compound of the invention to a subject in need of treatment. The instant pharmaceutical composition includes administration of a single pharmaceutical dosage formulation which contains both the NPY5 antagonist in combination with a second anti-obesity agent, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant pharmaceutical composition is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the NPY5 antagonist and the second anti-obesity agent is realized by the patient at substantially the same time. Such beneficial effect is preferably achieved when the target blood level concentrations of each active drug are maintained at substantially the same time. It is preferred that the combination of the NPY5 antagonist and the second anti-obesity agent be co-administered concurrently on a once-a-day dosing schedule; however, varying dosing schedules, such as the NPY5 antagonist once a day and the anti-obesity agent once, twice or more times per day, is also encompassed herein. A single oral dosage formulation comprised of both a NPY5 antagonist and a second anti-obesity agent is preferred. A single dosage formulation will provide convenience for the patient, which is an important consideration especially for patients with diabetes or obese patients who may be in need of multiple medications.

The term “subject”, as used herein refers to an animal, preferably a mammal, most preferably a human, who has been the object of treatment, observation or experiment.

The term “subject in need thereof” refers to a subject who is in need of treatment or prophylaxis as determined by a researcher, veterinarian, medical doctor or other clinician. In one embodiment, the subject in need of treatment is an obese mammal. In another embodiment, the subject in need of treatment is an obese human with one or more obesity-related co-morbidities. In another embodiment, the subject in need of treatment is an obese human without obesity-related co-morbidities.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of obesity or an obesity-related disorder in subjects as risk for obesity or the obesity-related disorder.

The magnitude of prophylactic or therapeutic dose of the active ingredients (e.g. NPY5 antagonist, anti-obesity agent) of the composition will, of course, vary with the nature of the severity of the condition to be treated and with the particular compound in the composition and its route of administration. It will also vary according to the age, weight and response of the individual patient. In general, the daily dose range of each compound in the combination lies within the range of from about 0.0001 mg/kg to about 100 mg/kg, preferably from about 0.001 mg/kg to about 50 mg/kg body weight of a subject in single or divided doses. On the other hand, it may be necessary to use dosages outside these limits in some cases.

For use where a composition for intravenous administration is employed, a suitable dosage range is from about 0.0001 mg/kg to about 50 mg/kg, preferably from 0.001 mg/kg to about 20 mg/kg of each compound in the composition per day.

In the case where an oral composition is employed, a suitable dosage range is, e.g. from about 0.001 mg/kg to about 100 mg/kg of each compound in the composition per day, preferably from about 0.01 mg to about 1000 mg per day. For oral administration, the compositions are preferably provided in the form of tablets containing from 0.01 mg to 1,000 mg, preferably 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound. In general, for treating obesity and/or an obesity related disorder, the NPY5 antagonist is administered at a daily dosage of from about 0.0001 mg/kg to about 100 mg/kg of body weight orally. More specifically, when treating obesity and/or obesity related disorders generally satisfactory results may be obtained when an NPY5 antagonist of Formula I, or a pharmaceutically acceptable salt or ester thereof, is administered at a daily oral dosage of from about 0.001 mg/kg to about 100 mg/kg, preferably from about 0.001 mg/kg to about 10 mg/kg of body weight, given in a single dose or in divided doses two to six times a day, or in sustained release form. A NPY5 antagonist of Formula II, or a pharmaceutically acceptable salt or ester thereof, may be administered at a daily oral dosage of from about 1 mg/kg to about 30 mg/kg of body weight, given in a single dose or in divided doses two to six times a day, or in sustained release form.

In general, for treating obesity and/or an obesity related disorder, the anti-obesity agent is administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form.

Leptin may be administered at a daily dosage of from about 0.01 mg/kg to about 20 mg/kg, preferably, from about 0.01 mg/kg to about 0.3 mg/kg, preferably injected in a single dose or in divided doses.

Metformin may be administered at a daily dosage of from about 0.01 mg/kg to about 100 mg/kg, preferably from about 1 mg/kg to about 50 mg/kg in a single dose or in divided doses 2 to 3 times per day, or in sustained release form; more preferably the daily dose is 500 mg, 850 mg, 1000 mg, 1500 mg, 2000 mg or 2550 mg orally given as a single dose or in divided doses 2 to 3 times per day.

Nalmefene may be administered at a daily dosage of from about 0.0001 mg/kg to about 10 mg/kg, preferably from about 0.001 to about 0.05 mg/kg.

Orlistat may be administered at a daily dosage of from about 20 mg to about 1200 mg, preferably from about 120 mg to 400 mg in a single dose or divided doses 2 to 3 times per day or in sustained release form; more preferably a 120 mg single dose 3 times per day, or in sustained release form.

Sibutramine may be administered at a daily dosage of from about 0.01 mg/kg to about 10 mg/kg, preferably from about 0.01 mg/kg to about 1 mg/kg in a single dose or in divided doses 2 to 3 times per day, or in sustained release form; more preferably the single daily dose of sibutramine is 5 mg, 10 mg, 15 mg, 20 mg or 30 mg orally.

Rimonabant may be administered at a daily dosage of from about 0.01 mg/kg to about 8 mg/kg, more preferably from about 0.3 mg/kg to about 3 mg/kg of body weight in a single dose or in divided doses 2 to 3 times per day, or in sustained release form.

Topiramate (Topamax®) may be administered at a daily dosage of from about 10 mg to about 1,600 mg per day, preferably from about 50 mg to about 400 mg per day in a single dose or in divided doses, or in sustained release form.

Zonasamide may be administered at a daily dosage of from about 10 mg to about 1,500 mg per day, preferably from about 100 mg to about 600 mg per day in a single dose or in divided doses, or in sustained release form. More preferably zonasamide may be administered at a daily dosage of from about 100 mg/d orally, with gradual increase to 400 mg/d and further increase to 600 mg/d for patients losing less than 5% of body weight at the end of 12 weeks.

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

The weight ratio of the NPY5 antagonist of Formula I or II to the second anti-obesity agent may be varied and will depend upon the effective dose of each ingredient. Generally, an effective dose of each will be used. Thus, for example, when a NPY 5 antagonist of Formula I or II is combined with a second anti-obesity agent, such as sibutramine, the weight ratio of the NPY5 antagonist of the Formula I or II to the sibutramine will generally range from about 1000:1 to about 1:1000, preferably about 200:1 to about 1:200. Compositions of a NPY5 antagonist of Formula I or II and other anti-obesity agents will generally also be within the aforementioned range, but in each case, an effective dose of each active ingredient should be used.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a NPY5 antagonist, additional active ingredient(s) such as a second anti-obesity agent, and pharmaceutically acceptable excipients.

Any suitable route of administration may be employed for providing a subject, especially a human, with an effective dosage of a composition of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a combination of a NPY5 antagonist and a second anti-obesity agent, as active ingredients or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. NPY5 antagonist or anti-obesity agent) can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g., NPY5 antagonist, anti-obesity agent) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active in the composition of the present invention (eg. NPY5 antagonist, anti-obesity agent) for the symptomatic adjustment of the dosage to the subject to be treated.

Exemplifying the invention is a pharmaceutical composition comprising a NPY5 antagonist and a second anti-obesity agent described above and a pharmaceutically acceptable carrier. Also exemplifying the invention is a pharmaceutical composition made by combining any of the NPY5 antagonists and anti-obesity agents described above and a pharmaceutically acceptable carrier. An illustration of the invention is a process for making a pharmaceutical composition comprising combining any of the NPY5 antagonists and anti-obesity agents described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Injectable Suspension (I.M.) | mg/mL |
|---|---|
| NPY5 antagonist of Formula I | 0.70 |
| Nalmefene | 1.0 |
| cyclodextrin | Q.S. ed to |
| (35% weight/volume) | 1 ml volume |
| glycerol | 63.05 |
| Water for injection to a total volume of | 1 mL |

| Tablet | mg/tablet |
|---|---|
| NPY5 antagonist of Formula I | 25 |
| Sibutramine | 10 |
| Microcrystalline Cellulose | 40.5 |
| Lactose | 111.5 |
| Croscarmellose Sodium | 5.0 |
| Hydroxypropylcellulose | 6.0 |
| Sodium Dodecyl Sulfate | 1.0 |
| Magnesium Stearate | 1.0 |
| | 200 |

| Capsule | mg/capsule |
|---|---|
| NPY5 antagonist of Formula I | 100 |
| Sibutramine | 10 |
| Lactose | 70 |
| Sodium Dodecyl Sulfate | 20 |
| | 200 |

| Aerosol | Per canister | |
|---|---|---|
| NPY5 antagonist of Formula I | 4 | mg |
| Sibutramine | 9 | mg |
| Lecithin, NF Liq. Conc. | 1.2 | mg |
| Trichlorofluoromethane, NF | 4.025 | g |
| Dichlorodifluoromethane, NF | 12.15 | g |

It will be understood that the scope of compositions of the compounds of this invention with other agents useful for treating or preventing obesity and obesity-related conditions includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

In Vivo Pair-Feeding Study with a NPY5 Antagonist

Materials and Methods

Compound A, is an orally active, selective NPY5 antagonist (Kanatani et al., 2000, Biochem. Biophys. Research Comm. 272:169-173). Male C57BL/6J mice (CLEA Japan Inc., 16 months old at the beginning of the drug administration) were used. Mice were given water and regular pellet chow (CE-2, CLEA Japan Inc.) ad libitum. They were kept in an animal room which was maintained at 23±2° C. temperature, 55±15% relative humidity and on a 12-hr light-dark cycle (7:00-19:00) during a quarantine and acclimatization period of 1 week.

Before the start of drug administration, mice were fed a MB diet (Oriental BioService Co., Tokyo, Japan) for about 9 to 10 months until the body weight gain reached plateau. After the body weight gain reached a plateau, the diet was change to a powder MHF diet. The powder MEW diet was given by powder feeder (small dishes). Diet and dishes were changed everyday, and daily food intake was measured. During this period, animals were orally administered vehicle (0.5% methylcellulose in distilled water) by gavage once-daily. After stable feeding was observed, the amount of new food was adjusted to daily food intake+0.3 g, to minimize the amount of spilled food. After the acclimation period, the MB diet-fed mice were divided into three groups to match average values of body weight (initial average body weight was 48 to 49 g). Each group was orally administered either vehicle or compound A at a dose of 100 mg/kg once-daily for 1 month, or vehicle by gavage. Food and body weight were measured. The administration was done one and half hours before the beginning of the dark period following the body weight measurement. Four days after drug administration, the pair-feeding was started. The pair-fed amount of food was calculated from the following equations:
Average F.I. inhibition of drug treated group (%)=(average F.I. from control group−average F.I. from drug treated group)/average F.I. from control group×100 Pair fed amount of food for each animal=each animal's average F.I. from pre period×(1−average F.I. inhibition of drug treated group (%))
Pair fed mice were given a diet calculated from above inhibition and pre-average F.I. value. The amount of food was divided to two meals and given around at 8:00 and 18:00 to avoid a long duration of fasting.
Rectal temperature was measured at 13:00-14:00 by insertion of thermo-probe in the rectum, at the 14, 22, 29, 34th day (10, 18, 25, 30th day for pair-fed group) after the start of treatment.
Results

FIG. 1 shows the body weight change, FIG. 2 shows the daily food intake and FIG. 3 shows the rectal temperature in compound A-treated or pair-fed DIO mice. Compound A at a dose of 100 mg/kg once-daily significantly reduced body weight, and mildly reduced food intake, throughout the treatment period. Inhibitory percentage of cumulative food intake in compound A-treated group was 12.5%. In the pair-fed group, the amount of food intake was almost the same as that in the compound A-treated group, and the inhibitory percentage of cumulative food intake was 11.9%. In the pair-fed group, the body weight was reduced similarly to that of the compound A-treated group only for the first few days, then the body weight reduction stopped and remained constant throughout the experiment. Rectal temperature in the pair-fed group was slightly, but significantly reduced throughout the experiment. On the other hand, the rectal temperature in the Compound A treated group was almost the same as that in the control group.

Since the weight reduction in the NPY5 antagonist-treated group exceeded the weight loss in the vehicle group, this example demonstrates that a moderate feeding reduction and another mechanism contributes to the anti-obese effect of a NPY5 antagonist. The reduction in body temperature in the pair-fed group is indicative of a reduction in metabolic rate, presumably as compensation for reduced food intake. A similar reduction in metabolic rate has been reported in people when dieting, which makes it more difficult for people to reduce their body weight (Grubbs, L., 1993, Nurse Practitioner, 18: 20-2,25-6,29). The results of rectal temperature measurements indicate that NPY5 treatment prevents a food restriction induced decrease in body temperature. This suggests that one of the mechanism of anti-obesity effect of a NPY5 antagonist is to prevent a decrease in metabolic rate that accompanies food restriction such as dieting.

### EXAMPLE 2

In Vivo Study of a Combination of a NPY5 Antagonist and Food Restriction

Materials and Methods

Male C57BL/6J mice (CLEA Japan Inc., 14 months old at the beginning of the drug administration) were used. Mice were given water and a regular pellet chow (CE-2, CLEA Japan Inc.) ad libitum. They were kept in an animal room which was maintained at 23±2° C. temperature, 55±15% relative humidity and on a 12-hr light-dark cycle (7:00-19:00) during a quarantine and acclimatization period of 1 week. Before the start of drug administration, mice were fed a MHF diet (Oriental BioService Co., Tokyo, Japan) for about 9 to 10 months until the body weight gain reached a plateau. After the body weight gain reached a plateau, the diet was changed to a powder MHF diet. The powder MHF diet was given by powder feeder (small dishes). Diet and dishes were changed everyday, and the daily food intake was measured. During this period, animals were orally administered vehicle (0.5% methylcellulose in distilled water) by gavage twice-daily. After stable feeding was observed, the amount of new food was adjusted to daily food intake+0.3 g, to minimize the amount of spilled food. After the acclimation period, the My diet-fed mice were divided into four groups to match the average values of body weight (initial average body weight was 49 to 51 g) and food intake (n=7-10). Two of the groups were orally administered either vehicle or compound B at a dose of 30 mg/kg twice-daily for 1.5 months by gavage, respectively. Another two groups were treated with vehicle or compound B and given 90% of the average baseline food intake of the each mouse during the acclimation period (10% food restriction). The administration was done one and half hours before the beginning of the dark period following body weight measurement and one and half hours after the beginning of the light period. The amount of food was divided into two meals and given around at 8:00 and 18:00 to avoid a long duration of fasting. Food and body weight were measured.

Results

FIG. 4 shows the body weight change, FIG. 5 shows the % inhibition of body weight in each group. Compound B at a dose of 30 mg/kg twice-daily significantly reduced body weight, and mildly reduced food intake, throughout the treatment period. Inhibitory percentage of cumulative food intake in the Compound B-treated group was 6.0%. Ten % food restriction alone reduced the body weight similarly to that of Compound B-treated group only the first few days. After that, the body weight stopped decreasing and changed in parallel to that of the control group. On the other hand, the combination of Compound B and 10% food restriction decreased weight than either treatment alone in the DIO model. These results suggest that the combination of a NPY5 antagonist and a second obesity agent will be effective for the treatment of obesity.

### EXAMPLE 3

In Vivo Study for Combination Therapy with a NPY5 Antagonist and a Second Anti-Obesity Agent

DIO mice are treated simultaneously with an effective dose of a NPY5 antagonist and an effective dose of a second anti-obesity agent.

Materials and Methods

Male C57BL/6J mice (CLEA Japan Inc., 12-16 months old at the beginning of the drug administration) are used. Mice are given water and regular pellet chow (CE-2, CLEA Japan Inc.) ad libitum. They are kept in an animal room which is maintained at 23±2° C. temperature, 55±15% relative humidity and on a 12-hr light-dark cycle (7:00-19:00) during a quarantine and acclimatization period of 1 week. Before the start of drug administration, mice are fed a MHF diet (Oriental BioService Co., Tokyo, Japan) for about 9 to 10 months until the body weight gain reaches a plateau. After the body weight gain reaches a plateau, the diet is changed to a powder MHF diet. The powder MHF diet is given by powder feeder (small dishes). Diet and dishes are changed everyday, and daily food intake is measured. During this period, animals are orally administered vehicle (0.5% methylcellulose in distilled water) by gavage once-daily. After the stable feeding is observed, the amount of new food is adjusted to daily food intake+0.3 g, to minimize the amount of spilled food. After the acclimation period, the MHF diet-fed mice are divided into four groups to match average values of body weight and food intake (n=7-10). Two of the groups are orally administered either vehicle or compound A at a dose of 100 mg/kg once-daily for 1.5 months by gavage, respectively. Another two groups are treated with vehicle or compound A and given an effective dose of a second anti-obesity agent. The administration is done one and half hours before the beginning of the dark period following the measurement of body weight. If the duration of action of the second anti-obesity agent is limited, it is dosed twice-daily. The administration is done one and half hours before the beginning of the dark period following body weight measurement and one and half hours after the beginning of the light period. The amount of food is divided into two meals and given at about 8:00 and 18:00 to avoid a long duration of fasting. Food and body weight are measured.

Effective anti-obesity combinations result in a greater body weight change when the NPY5 antagonist and the second anti-obesity agent are given together, than the body weight change seen with either compound is administered alone.

### EXAMPLE 4

Human Study for Combination Therapy with a NPY5 Antagonist and a Second Anti-Obesity Agent

Methods

800 people with a BMI>30 are advised to diet and increase their physical activity. After a two-week placebo run-in period, which includes a standardized program of diet, physical activity, and lifestyle changes, the patients are randomized into 4 treatment groups: placebo; an effective dose of a NPY5 antagonist, such as 1000 mg of Compound A; an effective dose of a second anti-obesity agent, such as 15 mg of sibutramine; and an effective dose of the NPY5 antagonist plus an effective dose of the second anti-obesity agent. The NPY5 antagonist is given in tablet form once or more per day, as previously determined to be effective. The second anti-obesity agent is given in tablet form once or more per day, as previously determined to be effective. When the second anti-obesity agent is sibutramine, capsules of sibutramine are given once per day. Patients are treated for 6 months, body weights are measured weekly, and appetite, hunger, satiety are measured weekly using standard questionnaires.

Effective anti-obesity combinations result in a greater body weight change when the NPY5 antagonist and the second anti-obesity agent are given together, than the body weight change seen with either compound is administered alone.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A composition comprising
(a) a NPY5 antagonist of formula I or II 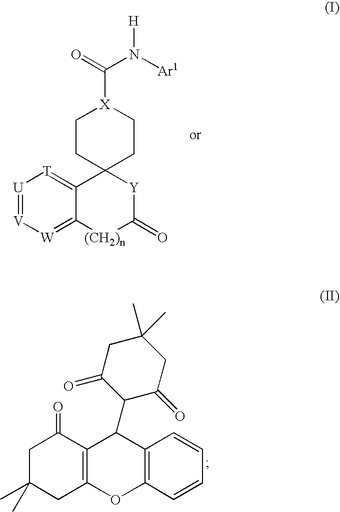
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy, and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen; and
(b) an antiobesity agent selected from the group consisting of:
(1) 5HT transporter inhibitor;
(2) NE transporter inhibitor;
(3) CB-1 antagonist/inverse agonist;
(4) Ghrelin antagonist;
(5) H3 antagonist/inverse agonist;
(6) MCH1R antagonist;
(7) MCH2R agonist/antagonist;
(8) NPY1 antagonist;
(9) leptin;
(10) leptin derivatives;
(11) opioid antagonist;
(12) orexin antagonist;
(13) BRS3 agonist;
(14) CCK-A agonist;
(15) CNTF;
(16) CNTF derivatives;
(17) GHS agonist;
(18) 5HT2C agonist;
(19) monoamine reuptake inhibitor;
(20) UCP-1, 2, and 3 activator;
(21) β3 agonist;
(22) thyroid hormone β agonist;
(23) PDE inhibitor;
(24) FAS inhibitor;
(25) DGAT1 inhibitor;
(26) DGAT2 inhibitor;
(27) ACC2 inhibitor;
(28) glucocorticoid antagonist;
(29) acyl-estrogens;
(30) lipase inhibitor;
(31) fatty acid transporter inhibitor;
(32) dicarboxylate transporter inhibitor;
(33) glucose transporter inhibitor;
(34) serotonin reuptake inhibitors;
(35) metformin; and
(36) topiramate;
and pharmaceutically acceptable salts and esters thereof.

2. The composition of claim 1 wherein the anti-obesity agent is selected from the group consisting of:
(1) acyl-estrogen;
(2) CB-1 antagonist/inverse agonist;
(3) opioid antagonist;
(4) monoamine reuptake inhibitor;
(5) lipase inhibitor;
(6) leptin;
(7) CNTF;
(8) CNTF derivatives;
(9) metformin; and
(10) topiramate;
and pharmaceutically acceptable salts and esters thereof.

3. The composition of claim 2 wherein the acyl-estrogen is selected from oleoyl-estrone, the monoamine reuptake inhibitor is selected from sibutramine, the CNTF derivative is selected from axokine, the lipase inhibitor is selected from orlistat, the CB-1 antagonist/inverse agonist is selected from rimonabant and the opioid antagonist is selected from nalmefene, and the pharmaceutically acceptable salts thereof.

4. (canceled)

5. (canceled)

6. (canceled)

7. (canceled)

8. (canceled)

9. (canceled)

10. (canceled)

11. (canceled)

12. The composition of claim 1 wherein the NPY5 antagonist is selected from the group consisting of a compound of formula I 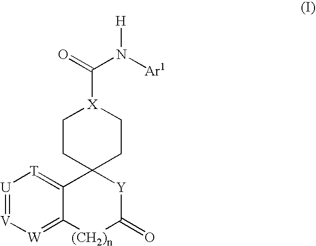
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy; and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen.

13. (canceled)

14. The composition of claim 12 wherein the NPY5 antagonist is selected from the group consisting of
(1) 3-oxo-N-(5-phenyl-2-pyrazinyl)-spiro[isobenzofuran-1(3H),4′-piperidine]-1′-carboxamide;
(2) 3-oxo-N-(7-trifluoromethylpyrido[3,2-b]pyridin-2-yl)spiro-[isobenzofuran-1(3H),4′-piperidine]-1′-carboxamide;
(3) N-[5-(3-fluorophenyl)-2-pyrimidinyl]-3-oxospiro-[isobenzofuran-1(3H),4′-piperidine]-1′-carboxamide;
(4) trans-3′-oxo-N-(5-phenyl-2-pyrimidinyl)spiro[cyclohexane-1,1′(3′H)-isobenzofuran]-4-carboxamide;
(5) trans-3′-oxo-N-[1-(3-quinolyl)-4-imidazolyl]spiro[cyclohexane-1,1′(3′H)-isobenzofuran]-4-carboxamide;
(6) trans-3-oxo-N-(5-phenyl-2-pyrazinyl)spiro[4-azaiso-benzofuran-1(3H),1′-cyclohexane]-4′-carboxamide;
(7) trans-N-[5-(3-fluorophenyl)-2-pyrimidinyl]-3-oxospiro[5-azaisobenzofuran-1(3H),1′-cyclohexane]-4′-carboxamide;
(8) trans-N-[5-(2-fluorophenyl)-2-pyrimidinyl]-3-oxospiro[5-azaisobenzofuran-1(3H),1′-cyclohexane]-4′-carboxamide;
(9) trans-N-[1-(3,5-difluorophenyl)-4-imidazolyl]-3-oxospiro[7-azaisobenzofuran-1(3H),1′-cyclohexane]-4′-carboxamide;
(10) trans-3-oxo-N-(1-phenyl-4-pyrazolyl)spiro[4-azaisobenzofuran-1(3H),1′-cyclohexane]-4′-carboxamide;
(11) trans-N-[1-(2-fluorophenyl)-3-pyrazolyl]-3-oxospiro[6-azaisobenzofuran-1(3H),1′-cyclohexane]-4′-carboxamide;
(12) trans-3-oxo-N-(1-phenyl-3-pyrazolyl)spiro[6-azaisobenzofuran-1(3H),1′-cyclohexane]-4′-carboxamide; and
(13) trans-3-oxo-N-(2-phenyl-1,2,3-triazol-4-yl)spiro[6-azaisobenzofuran-1(3H),1′-cyclohexane]-4′-carboxamide;
and pharmaceutically acceptable salts and esters thereof.

15. A composition comprising
(a) a NPY5 antagonist of formula I or II 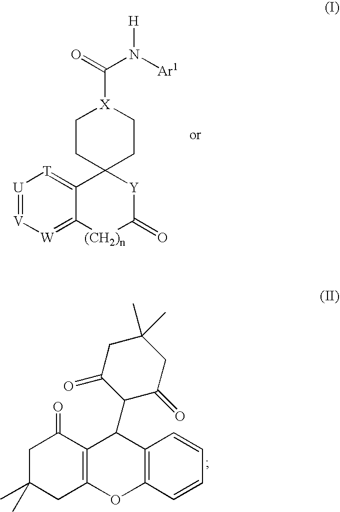
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy, and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen; and
(b) an anti-obesity agent selected from the group consisting of:
(1) aminorex;
(2) amphechloral;
(3) amphetamine;
(4) benzphetamine;
(5) chlorphentermine;
(6) clobenzorex;
(7) cloforex;
(8) clominorex;
(9) clortermine;
(10) cyclexedrine;
(11) dexfenfluramine;
(12) dextroamphetamine;
(13) diethylpropion;
(14) diphemethoxidine,
(15) N-ethylamphetamine;
(16) fenbutrazate;
(17) fenfluramine;
(18) fenisorex;
(19) fenproporex;
(20) fludorex;
(21) fluminorex;
(22) furfurylmethylamphetamine;
(23) levamfetamine;
(24) levophacetoperane;
(25) mazindol;
(26) mefenorex;
(27) metamfepramone;
(28) methamphetamine;
(29) norpseudoephedrine;
(30) pentorex;
(31) phendimetrazine;
(32) phenmetrazine;
(33) phentermine;
(34) phenylpropanolamine; and
(35) picilorex;
and pharmaceutically acceptable salts thereof.

16. A composition comprising
(a) a NPY5 antagonist selected from the group consisting of:
(1) 3-oxo-N-(5-phenyl-2-pyrazinyl)-spiro[isobenzofuran-1(3H),4′-piperidine]-1′-carboxamide;
(2) 3-oxo-N-(7-trifluoromethylpyrido[3,2-b]pyridin-2-yl)spiro-[isobenzofuran-1(3H),4′-piperidine]-1′-carboxamide;
(3) N-[5-(3-fluorophenyl)-2-pyrimidinyl]-3-oxospiro-[isobenzofuran-1(3H),4′-piperidine]-1′-carboxamide;
(4) trans-3′-oxo-N-(5-phenyl-2-pyrimidinyl)spiro[cyclohexane-1,1′(3′H)-isobenzofuran]-4-carboxamide;
(5) trans-3′-oxo-N-[1-(3-quinolyl)-4-imidazolyl]spiro[cyclohexane-1,1′(3′H)-isobenzofuran]-4-carboxamide;
(6) trans-3-oxo-N-(5-phenyl-2-pyrazinyl)spiro[4-azaiso-benzofuran-1(3H),1′-cyclohexane]-4′-carboxamide;
(7) trans-N-[5-(3-fluorophenyl)-2-pyrimidinyl]-3-oxospiro[5-azaisobenzofuran-1(3H),1′-cyclohexane]-4′-carboxamide;
(8) trans-N-[5-(2-fluorophenyl)-2-pyrimidinyl]-3-oxospiro[5-azaisobenzofuran-1(3H),1′-cyclohexane]-4′-carboxamide;
(9) trans-N-[1-(3,5-difluorophenyl)-4-imidazolyl]-3-oxospiro[7-azaisobenzofuran-1(3H),1′-cyclohexane]-4′-carboxamide;
(10) trans-3-oxo-N-(1-phenyl-4-pyrazolyl)spiro [4-azaisobenzofuran-1(3H),1′-cyclohexane]-4′-carboxamide;
(12) trans-N-[1-(2-fluorophenyl)-3-pyrazolyl]-3-oxospiro[6-azaisobenzofuran-1(3H),1′-cyclohexane]-4′-carboxamide;
(13) trans-3-oxo-N-(1-phenyl-3-pyrazolyl)spiro[6-azaisobenzofuran-1(3H),1′-cyclohexane]-4′-carboxamide; and
(14) trans-3-oxo-N-(2-phenyl-1,2,3-triazol-4-yl)spiro[6-azaisobenzofuran-1(3H),1′-cyclohexane]-4′-carboxamide;
and pharmaceutically acceptable salts and esters thereof; and
(b) a Mc4r agonist selected from the group consisting of:
(1) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl) pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl) pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl) pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl) pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl) pyrrolidin-3-yl]carbonyl}piperidin-4-yl)-4-fluoro-phenyl]-N-methylcarboxamide;
(7) 4-[2-(2-azetidin-1-yl-1 (S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl] 1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{((1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
and pharmaceutically acceptable salts thereof.

17. A composition comprising
(a) a NPY5 antagonist selected from the group consisting of:
(1) 3-oxo-N-(5-phenyl-2-pyrazinyl)-spiro[isobenzofuran-1(3H),4′-piperidine]-1′-carboxamide;
(2) 3-oxo-N-(7-trifluoromethylpyrido[3,2-b]pyridin-2-yl)spiro-[isobenzofuran-1(3H),4′-piperidine]-1′-carboxamide;
(3) N-[5-(3-fluorophenyl)-2-pyrimidinyl]-3-oxospiro-[isobenzofuran-1(3H),4′-piperidine]-1′-carboxamide;
(4) trans-3′-oxo-N-(5-phenyl-2-pyrimidinyl)spiro[cyclohexane-1,1′(3′H)-isobenzofuran]-4-carboxamide;
(5) trans-3′-oxo-N-[1-(3-quinolyl)-4-imidazolyl]spiro[cyclohexane-1,1′(3′ H)-isobenzofuran]-4-carboxamide;
(6) trans-3-oxo-N-(5-phenyl-2-pyrazinyl)spiro[4-azaiso-benzofuran-1(3H),1′-cyclohexane]-4′-carboxamide;
(7) trans-N-[5-(3-fluorophenyl)-2-pyrimidinyl]-3-oxospiro[5-azaisobenzofuran-1(3H),1′-cyclohexane]-4′-carboxamide;
(8) trans-N-[5-(2-fluorophenyl)-2-pyrimidinyl]-3-oxospiro[5-azaisobenzofuran-1(3H),1′-cyclohexane]-4′-carboxamide;
(9) trans-N-[1-(3,5-difluorophenyl)-4-imidazolyl]-3-oxospiro[7-azaisobenzofuran-1(3H),1′-cyclohexane]-4′-carboxamide;
(10) trans-3-oxo-N-(1-phenyl-4-pyrazolyl)spiro[4-azaisobenzofuran-1(3H),1′-cyclohexane]-4′-carboxamide;
(11) trans-N-[1-(2-fluorophenyl)-3-pyrazolyl]-3-oxospiro[6-azaisobenzofuran-1(3H),1′-cyclohexane]-4′-carboxamide;
(12) trans-3-oxo-N-(1-phenyl-3-pyrazolyl)spiro[6-azaisobenzofuran-1(3H),1′-cyclohexane]-4′-carboxamide; and
(13) trans-3-oxo-N-(2-phenyl-1,2,3-triazol-4-yl)spiro[6-azaisobenzofuran-1(3H),1′-cyclohexane]-4′-carboxamide;
and pharmaceutically acceptable salts and esters thereof, and
(b) a Mc4r agonist selected from the group consisting of:
(1) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide,
(2) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide,
(3) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(4) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide,
(5) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide, and
(6) 4-[2-(2-azetidin-1-yl-1 (S)-methyl-2-oxoethyl)-4-chlorophenyl]-1-{[(3S,4R)-1-tert-butyl-4-(2,4-difluorophenyl)pyrrolidin-3-yl]carbonyl}piperidine,
and pharmaceutically acceptable salts thereof.

18. A composition according to claim 1 further comprising a pharmaceutically acceptable carrier.

19. (canceled)

20. (canceled)

21. A method of preventing obesity in a subject at risk for obesity comprising administration to said subject
(a) a prophylactically effective amount of a NPY5 antagonist of Formula I or II: 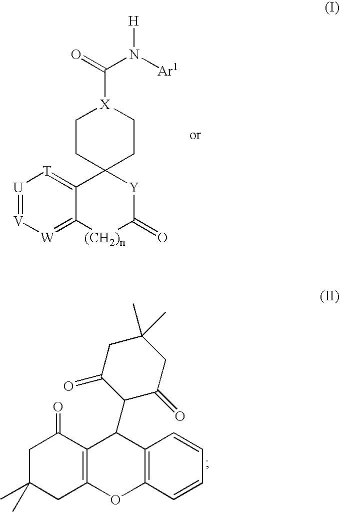
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
k) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy, and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen; and
(b) a prophylactically effective amount of an anti-obesity agent selected from the group consisting of:
(1) 5HT transporter inhibitor;
(2) NE transporter inhibitor;
(3) CB-1 antagonist/inverse agonist;
(4) Ghrelin antagonist;
(5) H3 antagonist/inverse agonist;
(6) MCH1R antagonist;
(7) MCH2R agonist/antagonist;
(8) NPY1 antagonist;
(9) leptin;
(10) leptin derivatives;
(11) opioid antagonist;
(12) orexin antagonist;
(13) BRS3 agonist;
(14) CCK-A agonist;
(15) CNTF;
(16) CNTF derivatives;
(17) GHS agonist;
(18) 5HT2C agonist;
(19) monoamine reuptake inhibitor;
(20) UCP-1, 2, and 3 activator;
(21) β3 agonist;
(22) thyroid hormone β agonist;
(23) PDE inhibitor;
(24) FAS inhibitor;
(25) DGAT1 inhibitor;
(26) DGAT2 inhibitor;
(27) ACC2 inhibitor;
(28) glucocorticoid antagonist;
(29) acyl-estrogens;
(30) lipase inhibitor;
(31) fatty acid transporter inhibitor;
(32) dicarboxylate transporter inhibitor;
(33) glucose transporter inhibitor;
(34) serotonin reuptake inhibitors;
(35) metformin; and
(36) topiramate;
and pharmaceutically acceptable salts and esters thereof.

22. A method of treating a subject having a disorder associated with excessive food intake comprising administration of
(a) a therapeutically effective amount of a NPY5 antagonist of Formula I or II: 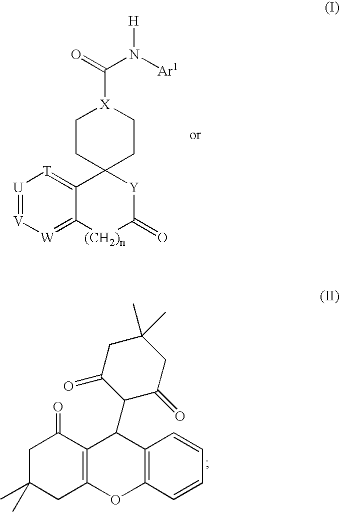
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy, and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen; and
(b) a therapeutically effective amount of an anti-obesity agent selected from the group consisting of:
(1) 5HT transporter inhibitor;
(2) NE transporter inhibitor;
(3) CB-1 antagonist/inverse agonist;
(4) Ghrelin antagonist;
(5) H3 antagonist/inverse agonist;
(6) MCH1R antagonist;
(7) MCH2R agonist/antagonist;
(8) NPY1 antagonist;
(9) leptin;
(10) leptin derivatives;
(11) opioid antagonist;
(12) orexin antagonist;
(13) BRS3 agonist;
(14) CCK-A agonist;
(15) CNTF;
(16) CNTF derivatives;
(17) GHS agonist;
(18) 5HT2C agonist;
(19) monoamine reuptake inhibitor;
(20) UCP-1, 2, and 3 activator;
(21) β3 agonist;
(22) thyroid hormone β agonist;
(23) PDE inhibitor;
(24) FAS inhibitor;
(25) DGAT1 inhibitor;
(26) DGAT2 inhibitor;
(27) ACC2 inhibitor;
(28) glucocorticoid antagonist;
(29) acyl-estrogens;
(30) lipase inhibitor;
(31) fatty acid transporter inhibitor;
(32) dicarboxylate transporter inhibitor;
(33) glucose transporter inhibitor;
(34) serotonin reuptake inhibitors;
(35) metformin; and
(36) topiramate;
and pharmaceutically acceptable salts and esters thereof,
to a subject in need of such treatment.

23. The method according to claim 22 wherein the disorder associated with excessive food intake is selected from obesity and an obesity-related disorder.

24. (canceled)

25. The method according to claim 23 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis;
gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; and acute lymphoblastic leukemia.

26. The method according to claim 25 wherein the obesity-related disorder is diabetes.

27. (canceled)

28. (canceled)

29. (canceled)

30. (canceled)

31. (canceled)

32. (canceled)

33. (canceled)

34. A method of preventing obesity in a subject at risk for obesity comprising administration to said subject
(a) a prophylactically effective amount of a NPY5 antagonist selected from the group consisting of:
(1) 3-oxo-N-(5-phenyl-2-pyrazinyl)-spiro[isobenzofuran-1(3H),4′-piperidine]-1′-carboxamide;
(2) 3-oxo-N-(7-trifluoromethylpyrido[3,2-b]pyridin-2-yl)spiro-[isobenzofuran-1(3H),4′-piperidine]-1′-carboxamide;
(3) N-[5-(3-fluorophenyl)-2-pyrimidinyl]-3-oxospiro-[isobenzofuran-1(3H),4′-piperidine]-1′-carboxamide;
(4) trans-3′-oxo-N-(5-phenyl-2-pyrimidinyl)spiro[cyclohexane-1,1′(3′H)-isobenzofuran]-4-carboxamide;
(5) trans-3′-oxo-N-[1-(3-quinolyl)-4-imidazolyl]spiro[cyclohexane-1,1 ′(3′H)-isobenzofuran]-4-carboxamide;
(6) trans-3-oxo-N-(5-phenyl-2-pyrazinyl)spiro[4-azaiso-benzofuran-1(3H),1′-cyclohexane]-4′-carboxamide;
(7) trans-N-[5-(3-fluorophenyl)-2-pyrimidinyl]-3-oxospiro[5-azaisobenzofuran-1(3H),1′-cyclohexane]-4′-carboxamide;
(8) trans-N-[5-(2-fluorophenyl)-2-pyrimidinyl]-3-oxospiro[5-azaisobenzofuran-1(3H),1′-cyclohexane]-4′-carboxamide;
(9) trans-N-[1-(3,5-difluorophenyl)-4-imidazolyl]-3-oxospiro[7-azaisobenzofuran-1(3H),1′-cyclohexane]-4′-carboxamide;
(10) trans-3-oxo-N-(1-phenyl-4-pyrazolyl)spiro[4-azaisobenzofuran-1(3H),1′-cyclohexane]-4′-carboxamide;
(11) trans-N-[1-(2-fluorophenyl)-3-pyrazolyl]-3-oxospiro[6-azaisobenzofuran-1(3H),1′-cyclohexane]-4′-carboxamide;
(12) trans-3-oxo-N-(1-phenyl-3-pyrazolyl)spiro[6-azaisobenzofuran-1(3H),1′-cyclohexane]-4′-carboxamide; and
(13) trans-3-oxo-N-(2-phenyl-1,2,3-triazol-4-yl)spiro[6-azaisobenzofuran-1(3H),1′-cyclohexane]-4′-carboxamide;
and pharmaceutically acceptable salts and esters thereof; and
(b) a prophylactically effective amount of a Mc4r agonist selected from the group consisting of:
(1) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl) pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl) pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl) pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl) pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl) pyrrolidin-3-yl]carbonyl}piperidin-4-yl)-4-fluoro-phenyl]-N-methylcarboxamide;
(7) 4-[2-(2-azetidin-1-yl-1 (S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1 (R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
and pharmaceutically acceptable salts thereof.

35. A The method of treating a subject having a disorder associated with excessive food intake comprising administration of
(a) a therapeutically effective amount of a NPY5 antagonist selected from the group consisting of:
(1) 3-oxo-N-(5-phenyl-2-pyrazinyl)-spiro[isobenzofuran-1(3H),4′-piperidine]-1′-carboxamide;
(2) 3-oxo-N-(7-trifluoromethylpyrido[3,2-b]pyridin-2-yl)spiro-[isobenzofuran-1(3H),4′-piperidine]-1′-carboxamide;
(3) N-[5-(3-fluorophenyl)-2-pyrimidinyl]-3-oxospiro-[isobenzofuran-1(3H),4′-piperidine]-1′-carboxamide;
(4) trans-3′-oxo-N-(5-phenyl-2-pyrimidinyl)spiro[cyclohexane-1,1′(3 ′H)-isobenzofuran]-4-carboxamide;
(5) trans-3′-oxo-N-[1-(3-quinolyl)-4-imidazolyl]spiro[cyclohexane-1,1′(3 ′H)-isobenzofuran]-4-carboxamide;
(6) trans-3-oxo-N-(5-phenyl-2-pyrazinyl)spiro[4-azaiso-benzofuran-1(3H),1′-cyclohexane]-4′-carboxamide;
(7) trans-N-[5-(3-fluorophenyl)-2-pyrimidinyl]-3-oxospiro[5-azaisobenzofuran-1(3H),1′-cyclohexane]-4′-carboxamide;
(8) trans-N-[5-(2-fluorophenyl)-2-pyrimidinyl]-3-oxospiro[5-azaisobenzofuran-1(3H),1′-cyclohexane]-4′-carboxamide;
(9) trans-N-[1-(3,5-difluorophenyl)-4-imidazolyl]-3-oxospiro[7-azaisobenzofuran-1(3H),1′-cyclohexane]-4′-carboxamide;
(10) trans-3-oxo-N-(1-phenyl-4-pyrazolyl)spiro [4-azaisobenzofuran-1(3H),1′-cyclohexane]-4′-carboxamide;
(11) trans-N-[1-(2-fluorophenyl)-3-pyrazolyl]-3-oxospiro[6-azaisobenzofuran-1(3H),1′-cyclohexane]-4′-carboxamide;
(12) trans-3-oxo-N-(1-phenyl-3-pyrazolyl)spiro[6-azaisobenzofuran-1(3H),1′-cyclohexane]-4′-carboxamide; and
(13) trans-3-oxo-N-(2-phenyl-1,2,3-triazol-4-yl)spiro[6-azaisobenzofuran-1(3H),1′-cyclohexane]-4′-carboxamide;
and pharmaceutically acceptable salts and esters thereof, and
(b) a therapeutically effective amount of a Mc4r agonist selected from the group consisting of:
(1) 2-[2-(1-{[(3S,4R)-1-tert-butyl-4-(2,4-difluorophenyl) pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl) pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl) pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl) pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl) pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl) pyrrolidin-3-yl]carbonyl}piperidin-4-yl)-4-fluoro-phenyl]-N-methylcarboxamide;
(7) 4-[2-(2-azetidin-1-yl-1 (S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1 (R)-methyl-2-oxoethyl)-4-chlorophenyl]-1-{[(3 S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3 S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3 S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
and pharmaceutically acceptable salts thereof;
to a subject in need of such treatment.

36. The method according to claim 35 wherein the disorder associated with excessive food intake is selected from obesity and an obesity-related disorder.

37. (canceled)

38. The method according to claim 36 wherein the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; and acute lymphoblastic leukemia.

39. The method according to claim 38 wherein the obesity-related disorder is diabetes.

40. (canceled)

41. (canceled)

42. (canceled)

43. (canceled)

44. (canceled)

45. (canceled)

46. (canceled)

47. A kit comprising at least one unit dosage of a prophylactically or therapeutically effective amount of a NPY5 antagonist of Formula I or II, 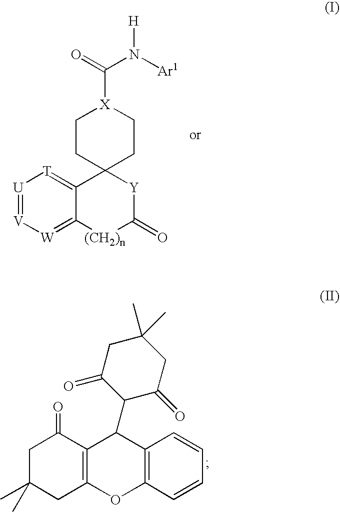
and pharmaceutically acceptable salts and esters thereof, and at least one unit dosage of a prophylactically or therapeutically effective amount of an anti-obesity agent, and pharmaceutically acceptable salts and esters thereof.

48. A method of maintaining weight loss in a subject comprising administration of
(a) a therapeutically effective amount of a NPY5 antagonist of Formula I or II: 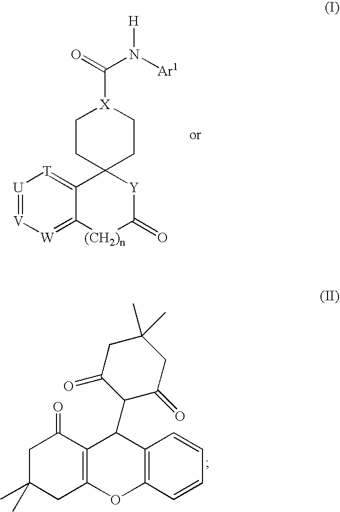
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of:
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy, and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen; and
(b) a therapeutically effective amount of an anti-obesity agent selected from the group consisting of:
(1) 5HT transporter inhibitor;
(2) NE transporter inhibitor;
(3) CB-1 antagonist/inverse agonist;
(4) Ghrelin antagonist;
(5) H3 antagonist/inverse agonist;
(6) MCH1R antagonist;
(7) MCH2R agonist/antagonist;
(8) NPY1 antagonist;
(9) leptin;
(10) leptin derivatives;
(11) opioid antagonist;
(12) orexin antagonist;
(13) BRS3 agonist;
(14) CCK-A agonist;
(15) CNTF;
(16) CNTF derivatives;
(17) GHS agonist;
(18) 5HT2C agonist;
(19) monoamine reuptake inhibitor;
(20) UCP-1, 2, and 3 activator;
(21) β3 agonist;
(22) thyroid hormone β agonist;
(23) PDE inhibitor;
(24) FAS inhibitor;
(25) DGAT1 inhibitor;
(26) DGAT2 inhibitor;
(27) ACC2 inhibitor;
(28) glucocorticoid antagonist;
(29) acyl-estrogens;
(30) lipase inhibitor;
(31) fatty acid transporter inhibitor;
(32) dicarboxylate transporter inhibitor;
(33) glucose transporter inhibitor;
(34) serotonin reuptake inhibitors;
(35) metformin;
(36) topiramate;
(37) zonisamide;
(38) aminorex;
(39) amphechloral;
(40) amphetamine;
(41) benzphetamine;
(42) chlorphentermine;
(43) clobenzorex;
(44) cloforex;
(45) clominorex;
(46) clortermine;
(47) cyclexedrine;
(48) dexfenfluramine;
(49) dextroamphetamine;
(50) diethylpropion;
(51) diphemethoxidine,
(52) N-ethylamphetamine;
(53) fenbutrazate;
(54) fenfluramine;
(55) fenisorex;
(56) fenproporex;
(57) fludorex;
(58) fluminorex;
(59) furfurylmethylamphetamine;
(60) levamfetamine;
(61) levophacetoperane;
(62) mazindol;
(63) mefenorex;
(64) metamfepramone;
(65) methamphetamine;
(66) norpseudoephedrine;
(67) pentorex;
(68) phendimetrazine;
(69) phenmetrazine;
(70) phentermine;
(71) phenylpropanolamine; and
(72) picilorex;
and pharmaceutically acceptable salts and esters thereof;
to a subject in need of such treatment.

49. A composition comprising
(a) a NPY5 antagonist of formula I or II 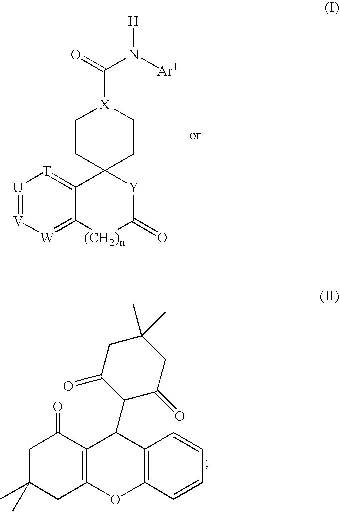
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl,
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy, and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen; and
(b) an anti-obesity agent selected from the group consisting of: zonisamide,
and pharmaceutically acceptable salts and esters thereof.

50. A method of treating a subject having a disorder associated with excessive food intake comprising administration of the composition of claim 49 to a subject in need thereof.

51. The method according to claim 50 wherein the disorder associated with excessive food intake is selected from obesity and an obesity-related disorder selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome: GH-deficient subjects; normal variant short stature; Turner's syndrome; and acute lymphoblastic leukemia.

52. (canceled)

53. (canceled)

54. (canceled)

55. A method of preventing obesity in a subject at risk for obesity comprising administration of the composition of claim 49 to said subject.
